(19) United States
(12) Patent Application Publication
(10) Pub. No.: US 2026/0123613 A1
(43) Pub. Date: May 7, 2026

(54) MOUSE MODEL OF DITRA DISEASE AND USES THEREOF

(71) Applicant: Regeneron Pharmaceuticals, Inc., Tarrytown, NY (US)

(72) Inventors: Andrew J. Murphy, Croton-on-Hudson, NY (US); Alexander O. Mujica, Elmsford, NY (US); Ka-Man Venus Lai, Seattle, WA (US); Sokol Haxhinasto, Brookfield, CT (US)

(73) Assignee: Regeneron Pharmaceuticals, Inc., Tarrytown, NY (US)

(21) Appl. No.: 19/363,935

(22) Filed: Oct. 21, 2025

Related U.S. Application Data
(63) Continuation of application No. 18/159,414, filed on Jan. 25, 2023, now Pat. No. 12,471,579.
Application No. 18/159,414 is a continuation of application No. 16/512,949, filed on Jul. 16, 2019, now Pat. No. 11,589,562.
(60) Provisional application No. 62/698,459, filed on Jul. 16, 2018.
(60) Provisional application No. 62/867,477, filed on Jun. 27, 2019.

(51) Int. Cl. A01K 67/0278; C07K 16/24; C07K 16/28; C12N 15/85

(52) U.S. Cl. CPC A01K 67/0278; C07K 16/244; C07K 16/2866; C12N 15/8509; A01K 2207/15; A01K 2217/072; A01K 2217/15; A01K 2227/105; A01K 2267/0368; C07K 2317/24; C07K 2317/76; C12N 2015/8527

(57) ABSTRACT

This disclosure relates to genetically modified rodent animals and rodent models of human diseases. More specifically, this disclosure relates to genetically modified rodents whose genome comprises a humanized Il1rl2 gene (coding for the IL1rl2 subunit of the IL-36R protein) and human IL-36α, β and γ ligand genes. The genetically modified rodents disclosed herein display enhanced skin and intestinal inflammation as a preclinical model of psoriasis and IBD, respectively, and serve as a rodent model of human DITRA disease.

### CROSS REFERENCE TO RELATED APPLICATIONS

This application is a continuation of U.S. patent application Ser. No. 18/159,414, filed Jan. 25, 2023, which is a continuation of U.S. patent application Ser. No. 16/512,949, filed Jul. 16, 2019, now U.S. Pat. No. 11,589,562, which claims the benefit of priority from U.S. Provisional Application No. 62/698,459, filed Jul. 16, 2018, and U.S. Provisional Application No. 62/867,477, filed Jun. 27, 2019, the entire contents of both of which are incorporated herein by reference.

### FIELD OF THE DISCLOSURE

This disclosure relates to genetically modified rodent animals and rodent models of human diseases. More specifically, this disclosure relates to genetically modified rodents whose genome comprises a humanized Il1rl2 gene (coding for the IL1rl2 subunit of an IL-36R) and human IL-36α, β and γ ligand genes. The genetically modified rodents disclosed herein display enhanced skin and intestinal inflammation in preclinical model of psoriasis and Inflammatory Bowel Disease (IBD), respectively, and may serve as a rodent model of human deficiency of the Interleukin-36R antagonist (DITRA) disease.

### INCORPORATION BY REFERENCE OF SEQUENCE LISTING

The sequence listing in XML, named as 35950ZY_10404US03_SequenceListing.xml of 86,501 bytes, created on Oct. 21, 2025, and submitted to the United States Patent and Trademark Office via Patent Center, is incorporated herein by reference.

### BACKGROUND ART

The Interleukin (IL)-36 subfamily consists of three agonists (IL-36α, β and γ, previously designated as IL1F6, IL1F8 and IL1F9, respectively) and one antagonist (IL36Ra) that signal through a heterodimeric receptor (IL-36R) leading to activation of nuclear factor-κB (NFκB) and mitogen-activated protein kinases (MAPKs). Similar to the classical IL-1 family members, IL-36 cytokines are involved in the initiation and regulation of immune responses. Members of the IL-36 family were first demonstrated to be predominantly expressed in squamous epithelial tissues, and, in particular, in psoriatic skin lesions. The link between IL-36R and epithelial-mediated diseases was confirmed in humans based on loss of function mutations in IL-36Ra leading to the diagnosis of Generalized Pustular Psoriasis (abbreviated “GPP”), now understood to be a species of GPP called deficiency of the Interleukin-36R antagonist (abbreviated “DITRA”). Elevated IL-36 expression was reported in GPP, palmoplantar pustular psoriasis (PPPP), inflammatory bowel disease (IBD), rheumatoid and psoriatic arthritis, asthma, chronic obstructive pulmonary disease, chronic kidney disease, and ichthyosis.

### SUMMARY OF THE DISCLOSURE

Disclosed herein are some embodiments of rodents genetically modified to comprise a humanized Il1rl2 gene encoding an Il1rl2 protein having an ectodomain substantially identical with a human IL1RL2 ectodomain, as well as human Il1f6, Il1f8 and Il1f9 genes encoding human IL1F6, IL1F8, and IL1F9, respectively. Il1rl2 is a subunit of the heterodimer IL-36R protein, which binds to three agonists: IL1F6, IL1F8 and IL1F9 (also known as IL-36α, β and γ, respectively). The rodents disclosed herein, while comprising a humanized Il1rl2 gene and expressing three human agonist ligand genes, maintain the endogenous IL-36Ra antagonist, where the endogenous IL-36Ra is about 20-fold less potent in inhibiting human IL-36R signaling. Such quadruple humanized rodents (e.g., humanized Il1rl2 and human IL1F6, IL1F8 and IL1F9) have been shown herein to display symptoms of DITRA patients where the mutation in IL-36Ra leads to an enhanced IL-36R signaling. Accordingly, genetically modified rodents, compositions and methods for making such rodents, and methods of using such rodents as a model for screening and developing therapeutic agents are provided therein.

In one aspect of some embodiments, disclosed herein is a genetically modified rodent whose genome comprises (1) a humanized Il1rl2 gene at an endogenous rodent Il1rl2 locus, wherein the humanized Il1rl2 gene encodes a humanized Il1rl2 protein that comprises an ectodomain substantially identical with the ectodomain of a human IL1RL2 protein; (2) a human IL1F6 gene at an endogenous rodent Il1f6 locus; (3) a human IL1F8 gene at an endogenous rodent Il1f8 locus; and (4) a human IL1F9 gene at an endogenous rodent Il1f9 locus.

In some embodiments, the humanized Il1rl2 gene in a rodent encodes a humanized Il1rl2 protein that comprises a transmembrane-cytoplasmic sequence substantially identical with the transmembrane-cytoplasmic sequence of the endogenous rodent Il1rl2 protein. In some embodiments, the humanized Il1rl2 gene in a rodent encodes a humanized Il1rl2 protein that comprises a signal peptide substantially identical with the signal peptide of the endogenous rodent Il1rl2 protein.

In some embodiments, the humanized Il1rl2 gene in a rodent encodes a humanized Il1rl2 protein comprising an ectodomain substantially identical with the ectodomain of a human IL1RL2 protein wherein the human IL1RL2 protein comprises the amino acid sequence as set forth in SEQ ID NO: 2.

In some embodiments, the humanized Il1rl2 gene in a rodent encodes a humanized Il1rl2 protein comprising an ectodomain wherein the ectodomain comprises amino acids 22-337 of SEQ ID NO: 7.

In some embodiments, the humanized Il1rl2 gene in a rodent encodes a humanized Il1rl2 protein that comprises the amino acid sequence as set forth in SEQ ID NO: 7.

In some embodiments, the humanized Il1rl2 gene in a rodent is operably linked to the endogenous rodent Il1rl2 promoter at the endogenous rodent Il1rl2 locus.

In some embodiments, the humanized Il1rl2 gene in a rodent results from a replacement of a genomic fragment of the endogenous rodent Il1rl2 gene at the endogenous rodent Il1rl2 locus with a nucleotide sequence of a human IL1RL2 gene.

In some embodiments, the nucleotide sequence of a human IL1RL2 gene is a genomic fragment of the human IL1RL2 gene that encodes substantially the ectodomain of the human IL1RL2 protein. In some embodiments, the genomic fragment of the human IL1RL2 gene comprises exons 3-8 of the human IL1RL2 gene.

In some embodiments, the genomic sequence of an endogenous rodent Il1rl2 gene remaining after a humanization replacement comprises the exons downstream of exon 8 of the endogenous rodent Il1rl2 gene. In some embodiments, the genomic sequence of the endogenous rodent Il1rl2 gene remaining after a humanization replacement comprises exons 1-2 of the endogenous rodent Il1rl2 gene.

In specific embodiments, the humanized Il1rl2 gene in a rodent disclosed herein comprises exons 1-2 of the endogenous rodent Il1rl2 gene, exons 3-8 of the human IL1RL2 gene, and the remaining exons downstream of exon 8 of the endogenous rodent Il1rl2 gene.

In some embodiments, the human IL1F6 gene in a rodent disclosed herein replaces the endogenous rodent Il1f6 gene at the endogenous rodent Il1f6 locus.

In some embodiments, the human IL1F8 gene in a rodent disclosed herein replaces the endogenous rodent I/1f8 gene at the endogenous rodent Il1f8 locus.

In some embodiments, the human IL1F9 gene in a rodent disclosed herein replaces the endogenous rodent Il1f9 gene at the endogenous rodent Il1f9 locus.

In some embodiments, a rodent disclosed herein is homozygous for one or more of the four humanized and/or human genes. In some embodiments, rodent disclosed herein is homozygous for each of the four humanized and/or human genes.

In some embodiments, a rodent disclosed herein displays shortened colon as compared to a wild type rodent. In some embodiments, a rodent disclosed herein displays enhanced inflammation in an experimentally induced inflammation model as compared to a wild type rodent. In some embodiments, the experimentally induced inflammation model is a skin inflammation model induced by imiquimod (IMQ). In some embodiments, the experimentally induced inflammation model is an intestinal inflammation model induced by dextran sulfate sodium (DSS) or oxazolone.

In some embodiments, a rodent disclosed herein is a mouse or a rat.

Further disclosed herein is an isolated cell or tissue from a rodent described herein.

In a further aspect of some embodiments, disclosed herein is a method of making a genetically modified rodent, the method comprising modifying a rodent genome to comprise (1) a humanized Il1rl2 gene at an endogenous rodent Il1rl2 locus, wherein the humanized Il1rl2 gene encodes a humanized Il1rl2 protein that comprises an ectodomain substantially identical with the ectodomain of a human IL1RL2 protein; (2) a human IL1F6 gene at an endogenous rodent Il1f6 locus; (3) a human IL1F8 gene at an endogenous rodent Il1f8 locus; and (4) a human IL1F9 gene at an endogenous rodent Il1f9 locus; and making a rodent comprising the modified genome.

In some embodiments, the rodent genome is modified by a process comprising (i) making a first rodent comprising a humanized Il1rl2 gene at the endogenous rodent Il1rl2 locus; (ii) making a second rodent comprising a human IL1F6 gene at an endogenous rodent Il1f6 locus, a human IL1F8 gene at the endogenous rodent Il1f8 locus, and a human IL1F9 gene at the endogenous rodent Il1f9 locus; and (iii) crossing the first rodent with the second rodent to obtain a modified rodent genome.

In some embodiments, the rodent comprising a humanized Il1rl2 gene at the endogenous rodent Il1rl2 locus is made by providing a rodent embryonic stem (ES) cell, inserting a nucleotide sequence of a human IL1RL2 gene into the rodent Il1rl2 locus of the rodent ES cell to form a humanized Il1rl2 gene at the rodent Il1rl2 locus, thereby obtaining a rodent ES cell comprising the humanized Il1rl2 gene, and making a rodent using the rodent ES cell comprising the humanized Il1rl2 gene.

In some embodiments, the nucleotide sequence of a human IL1RL2 gene being inserted replaces a genomic fragment of the rodent Il1rl2 gene at the rodent Il1rl2 locus. In some embodiments, the nucleotide sequence of a human IL1RL2 gene is a genomic fragment of the human IL1RL2 gene that encodes substantially the ectodomain of the human IL1RL2 protein. In some embodiments, the genomic fragment of the human IL1RL2 gene comprises exons 3-8 of the human IL1RL2 gene. In some embodiments, the genomic sequence of the endogenous rodent Il1rl2 gene remaining after the replacement comprises the exons downstream of exon 8 of the endogenous rodent Il1rl2 gene. In some embodiments, the genomic sequence of the endogenous rodent Il1rl2 gene remaining after a humanization replacement comprises exons 1-2 of the endogenous rodent Il1rl2 gene. In some embodiments, the humanized Il1rl2 gene formed as a result of a humanization replacement comprises exons 1-2 of the endogenous rodent Il1rl2 gene, exons 3-8 of the human IL1RL2 gene, and the remaining exons downstream of exon 8 of the endogenous rodent Il1rl2 gene.

In some embodiments, a rodent comprising a human IL1F6 gene, a human IL1F8 gene, and a human IL1F9 gene is made by: providing a rodent embryonic stem (ES) cell; inserting a human IL1F6 gene into the rodent Il1f6 locus of the rodent ES cell, a human IL1F8 gene into the rodent Il1f8 locus of the rodent ES cell, and a human IL1F9 gene into the rodent Il1f9 locus of the rodent ES cell, thereby obtaining a rodent ES cell comprising the human IL1F6 gene, the human IL1F8 gene, and the human IL1F9 gene; and making a rodent using the rodent ES cell comprising the human IL1F6 gene, the human IL1F8 gene, and the human IL1F9 gene.

In some embodiments, a human IL1F6 gene, a human IL1F8 gene, and a human IL1F9 gene, are provided in a contiguous nucleic acid molecule.

In some embodiments of the methods, the human IL1F6 gene replaces the endogenous rodent Il1f6 gene at the endogenous rodent Il1f6 locus.

In some embodiments of the methods, the human IL1F8 gene replaces the endogenous rodent Il1f8 gene at the endogenous rodent Il1f8 locus.

In some embodiments of the methods, the human IL1F9 gene replaces the endogenous rodent Il1f9 gene at the endogenous rodent Il1f9 locus.

In another aspect of some embodiments, disclosed herein is a rodent embryonic stem (ES) cell comprising (1) a humanized Il1rl2 gene at an endogenous rodent Il1rl2 locus, wherein the humanized Il1rl2 gene encodes a humanized Il1rl2 protein that comprises an ectodomain substantially identical with the ectodomain of a human IL1RL2 protein; (2) a human IL1F6 gene at an endogenous rodent Il1f6 locus; (3) a human IL1F8 gene at an endogenous rodent Il1f8 locus; and (4) a human IL1F9 gene at an endogenous rodent Il1f9 locus. In some embodiments, such ES cell can be made by methods disclosed herein. In some embodiments, use of such ES cell to make a rodent is also disclosed.

In still another aspect of some embodiments, disclosed herein are targeting nucleic acid constructs useful for modifying a rodent genome (e.g., in a rodent ES cell) for making a modified rodent.

In some embodiments, disclosed herein is a targeting nucleic acid construct comprising a nucleotide sequence of a human IL1RL2 gene, flanked by 5′ and 3′ rodent nucleotide sequences capable of mediating homologous recombination and integration of the nucleotide sequence of the human IL1RL2 gene into a rodent Il1rl2 locus to form a humanized Il1rl2 gene, wherein the humanized Il1rl2 gene encodes a humanized Il1rl2 protein that comprises an ectodomain substantially identical with the ectodomain of a human IL1RL2 protein.

In some embodiments, disclosed herein is a targeting nucleic acid construct, comprising a contiguous nucleic acid sequence which comprises: a human IL1F6 gene, a human IL1F8 gene, and a human IL1F9 gene, wherein the contiguous nucleic acid sequence is flanked by 5′ and 3′ rodent nucleotide sequences capable of mediating homologous recombination and integration of the contiguous nucleic acid sequence into the rodent locus encompassing the rodent Il1f6 gene, the rodent Il1f8 gene, and the rodent Il1f9 gene.

In a further aspect of some embodiments, disclosed herein is use of a rodent disclosed herein as a rodent model of human diseases associated with deregulated IL-36 signaling. As non-limiting examples of the embodiments disclosed herein, the rodent can be used to study the pathology and molecular basis of human diseases associated with deregulated IL-36 signaling (such as, but not limited to, DITRA), or to screen, test and develop therapeutic compounds useful for treating such diseases.

In still a further aspect of some embodiments, disclosed herein is a method of assessing the therapeutic efficacy of a candidate compound for treating a disease associated with deregulated IL-36 signaling, comprising administering an agent to a rodent disclosed herein to induce inflammation, administering a candidate compound to the rodent, and determining whether the candidate compound inhibits and/or decreases induced inflammation.

In some embodiments, the agent administered to induce inflammation is DSS or oxazolone, which induces intestinal inflammation in the rodent. In some embodiments, the agent administered to induce inflammation is IMQ which induces skin inflammation in the rodent.

In some embodiments, a candidate compound is administered to the rodent before, during, or after the administration of an agent that induces inflammation. In some embodiments, a candidate compound can be a small molecule compound, a nucleic acid inhibitor, or an antigen-binding protein such as an antibody. In some embodiments, a candidate compound is an antibody that specifically binds human IL-36R.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A-1D show embodiments of an exemplary strategy for humanization of mouse Il1rl2. FIG. 1A shows a diagram, not to scale, of the genomic organization of mouse Il1rl2 and human IL1RL2 genes. Exons are represented by thin bars placed across the genomic sequences. A mouse genomic fragment of about 25,324 base pairs (bp) to be deleted and a human genomic fragment of about 32,389 bp to be inserted are indicated. Locations of probes used in an assay described in Example 1 are indicated. FIG. 1B illustrates, not to scale, an exemplary modified BAC vector for humanization of an endogenous mouse Il1rl2 gene, along with the junction sequences at the bottom (SEQ ID NOS: 26-28), to achieve (i) replacement of mouse Il1rl2 exons 3-8 and intervening introns, along with the 3′ 155 bp of mouse intron 2 and the 5′ 642 bp of mouse intron 8 with human IL1RL2 exons 3-8 and intervening introns, as well as the 3′ 346 bp of human intron 2 and 5′ 1101 bp of human intron 8, and (ii) an insertion of a loxP-hUb1-em7-Neo-pA-mPrm1-Crei-loxP cassette (4996 bp) downstream of the insertion of the human genomic fragment in intron 8. FIG. 1C illustrates, not to scale, a humanized Il1rl2 allele after the neomycin cassette has been deleted, along with the junction sequences at the bottom (SEQ ID NOS: 26 and 29). FIG. 1D sets forth a sequence alignment of a mouse Il1rl2 protein (SEQ ID NO: 4), a human IL1RL2 protein (SEQ ID NO: 2), and a humanized Il1rl2 protein (SEQ ID NO: 7).

FIGS. 2A-2D show embodiments of an exemplary strategy for replacing mouse Il1f8, Il1f9, and Il1f6 with human IL1F8, IL1F9, and IL1F6. FIG. 2A shows a diagram, not to scale, of the genomic organization of mouse Il1f8, Il1f9, and Il1f6 genes and human IL1F8, IL1F9, AND IL1F6 genes. Exons are represented by thin bars placed across the genomic sequences. A mouse genomic fragment of about 76,548 bp to be deleted and a human genomic fragment of about 88,868 bp to be inserted are indicated. Locations of probes used in an assay described in Example 1 are indicated. FIG. 2B illustrates, not to scale, an exemplary modified BAC vector for replacement of endogenous mouse Il1f8, Il1f9, and Il1f6 genes, along with the junction sequences (SEQ ID NOS: 30-32) at the bottom, to achieve (i) replacement of coding sequences and untranslated regions (UTR), as well as intergenic sequence of mouse I/1f8, Il1f9, and Il1f6 with the corresponding human sequence of IL1F8, IL1F9, and IL1F6; and (ii) an insertion of a loxP-hUb1-em7-Hygro-pA-mPrm1-Crei-loxP cassette (5,218 bp) downstream of the insertion of the human genomic fragment. FIG. 2C illustrates, not to scale, a humanized locus after the hygromycin cassette has been deleted, along with the junction sequences at the bottom (SEQ ID NOS: 30 and 33). FIG. 2D sets forth sequence alignments of mouse Il1f6 (SEQ ID NO: 11) and human IL1F6 (SEQ ID NO: 9) proteins; of mouse Il1f8 (SEQ ID NO: 17) and human IL1F8 (SEQ ID NO: 15) proteins; and mouse Il1f9 (SEQ ID NO: 23) and human IL1F9 (SEQ ID NO: 21) proteins.

FIGS. 3A-3E show embodiments of the present invention, showing enhanced IMQ-induced skin inflammation in humanized DITRA-like mice. DITRA-like (also abbreviated as “DITRA” in the figures herein) and wild-type (WT) mice were treated with topical application of IMQ for 4 consecutive days. On day 5, back skin samples were collected for subsequent histopathological evaluation and qRT-PCR. (3A) Representative skin appearance of DITRA-like and WT mice at day 5 topically treated with Vaseline (“Vas”, control) or IMQ-containing cream (Aldara) for 4 consecutive days. (3B) Representative haematoxylin and eosin (“H&E”) staining of Vaseline- and IMQ-treated skin from DITRA-like and WT mice. (3C) mRNA expression of pro-inflammatory molecules in the skin of DITRA and WT mice treated daily with Vaseline or IMQ-containing cream (n=10 per each group). After topical application of IMQ for 4 consecutive days, on day 5, back skin samples were collected from Il1rl2 single humanized mice (“1H”), DITRA-like mice and wild type mice for subsequent histopathological evaluation and qRT-PCR. In each group of bars from left to right are: WT Vas, DITRA Vas, WT IMQ, and DITRA IMQ. (3D) Representative skin appearance of 1H, DITRA-like and WT mice at day 5 topically treated with Vaseline (control) or IMQ-containing cream (Aldara) for 4 consecutive days. (3E) mRNA expression of pro-inflammatory molecules in the skin of 1H, DITRA and WT mice treated daily with Vaseline or IMQ-containing cream (n=10 per each group). In each group of bars from left to right are: WT Vas, 1H Vas, DITRA-LIKE Vas, WT IMQ, 1H IMQ, and DITRA-LIKE IMQ.

FIGS. 4A-4B. In some embodiments, DITRA-like mice display shortened colons at steady state. (4A) Representative pictures of colons isolated from DITRA-like mice and their co-housed IL-36R KO and WT littermates at 3 and 10 months. (4B) Quantified colon length of 3 and 10 month old DITRA-like mice compared to their co-housed IL-36R KO and WT littermates (n=6 per each group). Error bars represent mean±SD.

FIGS. 5A-5E. In some embodiments, DITRA-like mice exhibit a defect in mucosal healing in DSS-induced chronic colitis. DITRA-like mice (n=16) and their co-housed WT littermate controls (n=14) were subjected to chronic DSS-induced colitis by administrating 2.5% DSS for 7 days followed by water for 11 days and 1.5% DSS for 4 days followed by water for 5 days for a total of 27 days. Control mice (n=5) received regular water. (5A) Body weight loss in DITRA-like mice and their WT co-housed littermate controls calculated as the percent difference between the original and actual body weight on any particular day. (5B) Colon length in water- and DSS-treated DITRA-like and WT mice. (5C) Haematoxylin and eosin (H&E) staining and pathology score of the colon of water- and DSS-treated DITRA-like and WT mice. (5D) Myeloperoxidase (MPO) activity in colon homogenates in water- and DSS-treated DITRA-like and WT mice. (5E) Levels of pro-inflammatory cytokines in colon homogenates in water- and DSS-treated DITRA-like and WT mice. Data are representative of at least three independent experiments. Error bars represent mean±SD. In each group of bars from left to right are: WT/water, DITRA/water, WT/DSS, and DITRA/DSS.

FIGS. 6A-6B. In some embodiments, human IL-36R antagonism with REGN anti-human IL-36R antibody ameliorates IMQ-induced skin inflammation in DITRA-like mice at prophylactic dosing. Skin on the back of DITRA-like mice was shaved 3 days before starting the IMQ application, and the skin was topically treated with Vaseline (control) or IMQ-containing cream (Aldara) for 4 consecutive days. PBS, αhIL-36R mAb and hIgG4 Isotype control were subcutaneously injected at 10 mg/mL on day-3 and 1 (n=9 for each treatment group). (6A) H&E histological sections and pathology score of the back skin isolated from DITRA-like mice on day 5. (6B) Levels of pro-inflammatory cytokines in the skin homogenates of DITRA-like mice on day 5 treated with daily applications of IMQ and injections of PBS, αIL-36R mAb and Isotype control on day-3 and 1. Data are representative of three independent experiments. Error bars represent mean±SD. In each group of bars from left to right are: PBS, αhIL-36R mAb, and hIgG4 isotype control.

FIGS. 7A-7C. In some embodiments, therapeutic administration of anti-human IL-36R antibody ameliorates chronic IMQ-induced skin inflammation in DITRA-like mice. Skin on the back of DITRA-like mice was shaved 3 days before starting the administration, and the skin was topically treated with Vaseline (control) or IMQ-containing cream (Aldara) for two rounds (5 consecutive days with the application for a first round, followed by 2 days without the application, then a second round of 4 consecutive days with the application for a total of 11 days). PBS, anti-human IL-36R antibody and hIgG4 Isotype control were subcutaneously injected at 10 mg/mL on days 5 and 9 (n=10 for each treatment group). (7A) Skin thickness was measured in DITRA-like mice on day 12. Thickness is presented in μm. (7B) H&E histological sections and pathology score of the skin from DITRA-like mice on day 12. (7C) Levels of pro-inflammatory cytokines were measured in the skin homogenates of DITRA-like mice on day 12. Data are representative of two independent experiments. Error bars represent mean±SD. In each group of bars from left to right are: phosphate buffered saline (PBS), αhIL-36R mAb, and hIgG4 isotype control.

FIGS. 8A-8E. In some embodiments, therapeutic administration of anti-human IL-36R antibody, similarly to IL-12p40 blockade, ameliorates DSS-induced chronic inflammation in DITRA-like mice and rescues their inability to heal from DSS-induced mucosal damage. DITRA-like mice (n=45) were subjected to chronic DSS-induced colitis by administrating 3% DSS for 7 days and 2% DSS for 13 days followed by water for the total of 30 days. Control mice (n=5) received regular water. PBS (n=11), αIL-36R mAb (n=11), hIgG4 Isotype control (n=6), αmIL-12p40 (BioxCell) (n=11) and rat IgG2a Isotype control (n=6) were intraperitoneally injected at 10 mg/mL on day 7, 10, 14, 17, 21 and 24. (8A) Body weight loss in DITRA-like mice calculated as the percent difference between the original and actual body weight on any particular day. (8B) Levels of fecal lipocalin-2 (Lcn2) in DITRA-like mice measured on day 0, 7, 17, 23 and 30 throughout the chronic colitis. (8C) Colon length in DITRA-like mice on day 30. (8D) Myeloperoxidase (MPO) activity in colon homogenates of DITRA-like mice at day 30. (8E) Levels of pro-inflammatory cytokines in colon homogenates in DITRA-like mice on day 30. Data are representative of two independent experiments. Error bars represent mean±SD. In each group of bars from left to right are: PBS/water, PBS/DSS, αhIL-36R/DSS, hIgG4 isotype control/DSS, αmIL-12p40/DSS, and rat IgG2a isotype control/DSS.

FIGS. 9A-9C. Human IL-36R antagonism ameliorates oxazolone-induced colitis in DITRA-like mice. DITRA-like mice were pre-sensitized with 3% solution of oxazolone dissolved in 100% Ethanol and intrarectally administered with 1.5% oxazolone and vehicle (50% Ethanol) on days 5, 6 and 7. Mice were intraperitoneally injected with PBS, anti-human IL-36R mAb and hIgG4 isotype control on days 2, 5 and 7 after pre-sensitization. (A) Body weight of PBS-, anti-human IL-36R mAb- and hIgG4 control-treated DITRA-like mice during oxazolone administration. * p<0.05 from PBS-treated group. (B) Colon length measured on day 8 in DITRA-like mice. (C) Levels of proinflammatory cytokines in colon homogenates in oxazolone- and vehicle-treated DITRA-like mice injected with PBS, anti-human IL-36R mAb and hIgG4 isotype control. *—represents significance from PBS-treated group, #-significance from isotype-treated group. Data are representative of two independent experiments with 5 mice per group. Error bars represent mean±SEM. *p<0.05, **p<0.005, ***p<0.0005, ****p<0.00001.

### DETAILED DESCRIPTION

Disclosed herein are rodents (such as, but not limited to, mice) genetically modified to comprise a humanized Il1rl2 gene (encoding a humanized IL1RL2 protein having an ectodomain substantially identical with a human IL1RL2 ectodomain) and human genes coding for human IL-36α, β, γ ligands, while keeping the IL-36Ra antagonist endogenous which is 20-fold less potent in inhibiting human IL-36R signaling. Genetically modified rodents disclosed herein have been shown to display symptoms of DITRA patients where the mutation in IL-36Ra leads to an enhanced IL-36R signaling. Therefore, the genetically modified rodents disclosed herein can serve as a novel functional model of DITRA suitable for testing candidate therapeutic agents for treating DITRA and related disorders. The various embodiments are further described below.

### Genetically Modified Rodents: Quadruple Humanization

In one aspect of some embodiments, this disclosure provides rodent animals that comprise in their genome a humanized Il1rl2 gene encoding a humanized IL1RL2 protein (a subunit of the IL-36R), and human genes encoding human IL-36α, β, and γ ligands. Such rodents are also referred to herein as quadruple humanized rodents (i.e., 4H or DITRA-like).

The term “humanized”, when used in the context of nucleic acids or proteins, includes nucleic acids or proteins of a rodent whose structures (i.e., nucleotide or amino acid sequences) have been modified, in whole or in part, to include structures found in corresponding human nucleic acids or proteins. A rodent containing a humanized gene or expressing a humanized protein is a “humanized” rodent.

In some embodiments, rodents of this disclosure include, as non-limiting examples, a mouse, a rat, and a hamster. In some embodiments, rodents of this disclosure include, as non-limiting examples, a mouse and a rat. In some embodiments, a rodent is selected from the superfamily Muroidea. In some embodiments, a rodent of this disclosure is from a family selected from Calomyscidae (e.g., mouse-like hamsters), Cricetidae (e.g., hamster, New World rats and mice, voles), Muridae (true mice and rats, gerbils, spiny mice, crested rats), Nesomyidae (climbing mice, rock mice, with-tailed rats, Malagasy rats and mice), Platacanthomyidae (e.g., spiny dormice), and Spalacidae (e.g., mole rates, bamboo rats, and zokors). In some embodiments, a rodent of this disclosure is selected from a true mouse or rat (family Muridae), a gerbil, a spiny mouse, and a crested rat. In some embodiments, a mouse of this disclosure is from a member of the family Muridae.

### IL-1RL2 Humanization

IL-36R is a member of the interleukin 1 receptor family and is a heterodimer that consists of a receptor subunit named IL1RL2 (also known as IL-1Rrp2) and a co-receptor subunit Interleukin-1 receptor accessory protein, IL-1RAcP (Garlanda C et al., Immunity 39, 1003-1018 (2013); Towne J E et al., J. Biol. Chem. 279, 13677-13688 (2004), which are incorporated by reference in their entireties). The receptor (IL-36R) can recognize and bind three different agonists, IL-36α, IL-36β, and IL-36γ (also known as IL1F6, IL1F8, and IL1F9), to induce the expression of inflammatory cytokines, as well as an antagonist, IL-36Ra, which binds to IL-36R and decreases the expression of inflammatory cytokines.

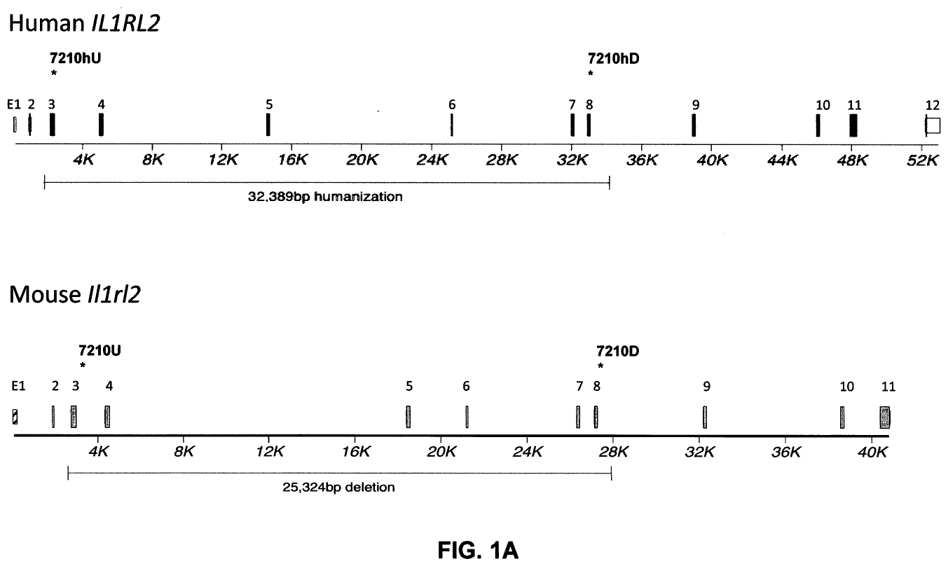

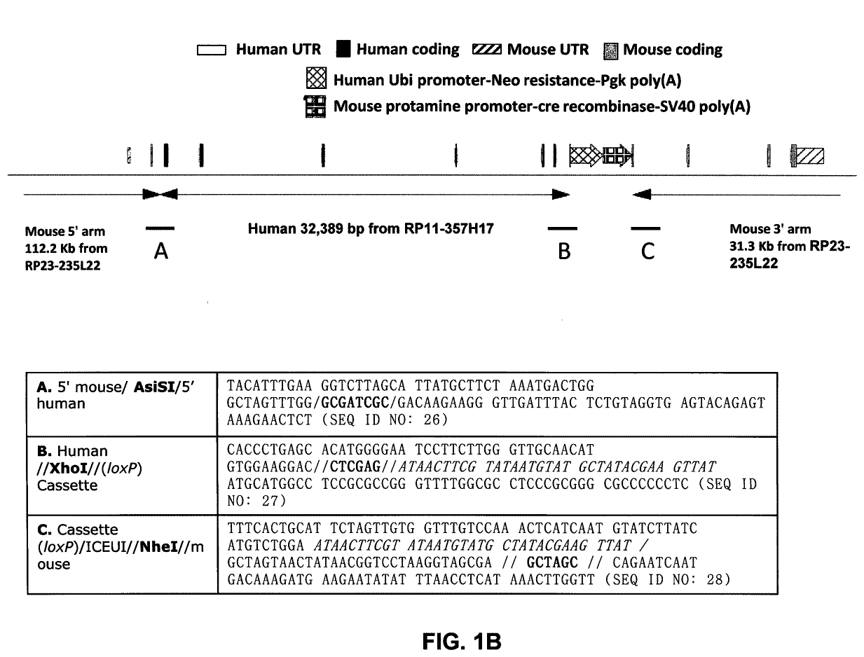

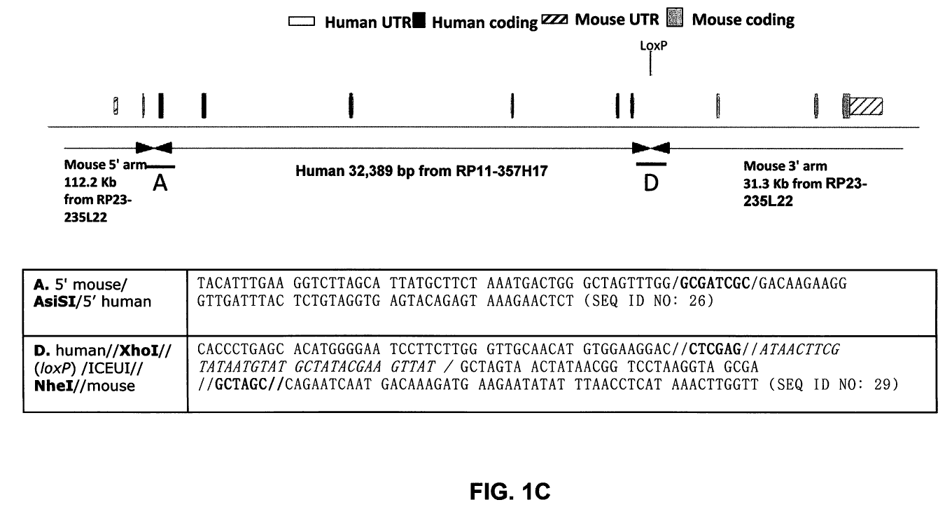

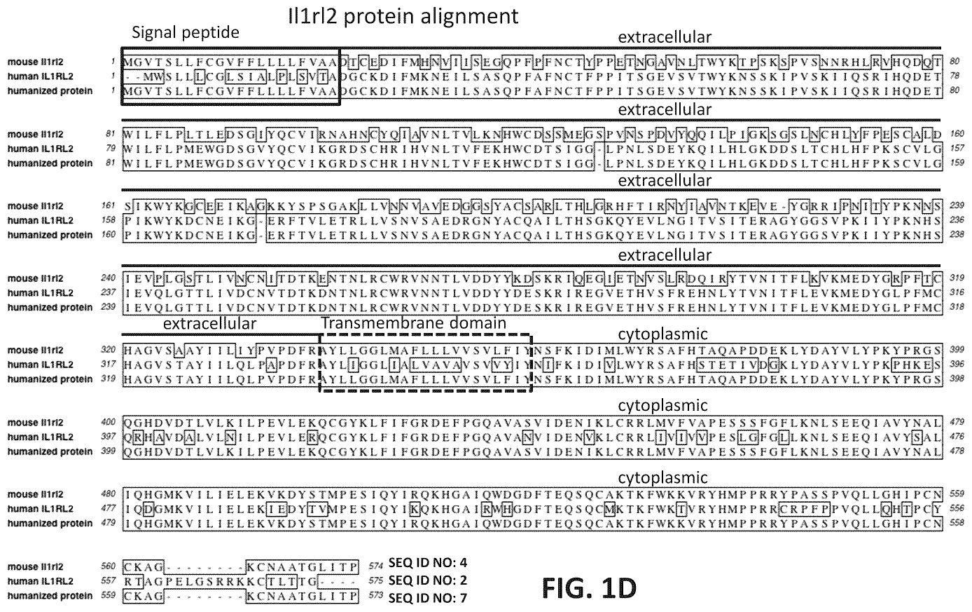

IL1RL2 contains a signal peptide, an extracellular domain (“ECD” or “ectodomain”), a transmembrane domain and an intracellular or cytoplasmic domain. See FIG. 1D, for example. Exemplary IL1RL2 sequences, including human, mouse, rat, and humanized Il1rl2 nucleic acid and protein sequences, are disclosed in the Sequence Listing and are summarized in the following table.

TABLE 1

| SEQ | | |
|---|---|---|
| ID NO | Description | Features |
| 1 | Homo sapiens IL1RL2 | Length: 2628 bp |
| | transcript variant 1 mRNA, | CDS: 127-1854 |
| | NM_003854.3 | 12 Exons: 1-114, 115-184, |
| | | 185-419, 420-615, 616-775, |
| | | 776-850, 851-980, 981-1117, |
| | | 1118-1261, 1262-1423, |
| | | 1424-1804, 1805-2615. |
| 2 | Homo sapiens IL1RL2 | Length: 575 aa |
| | protein, NP_003845.2 | Signal peptide: 1-19 |
| | | Mature: 20-575 |
| | | Ectodomain: 20-335 |
| 3 | Mus musculus Il1rl2 | Length: 4072 bp |
| | mRNA, NM_133193.4 | CDS: 238-1962 |
| | | 11 Exons: 1-231, 232-301, |
| | | 302-536, 537-735, 736-898, |
| | | 899-970, 971-1100, 1101-1237, |
| | | 1238-1381, 1382-1543, |
| | | 1544-4072. |
| 4 | Mus musculus Il1rl2 | Length: 574 aa |
| | Protein NP_573456.1 | Signal peptide: 1-21 |
| | | Mature: 22-574 |
| | | Ectodomain: 22-338 |
| 5 | Rattus norvegicus Il1rl2 | Length: 2044 bp |
| | mRNA, NM_133575.1 | CDS: 89-1774 |
| 6 | Rattus norvegicus Il1rl2 | Length: 561 aa |
| | Protein NP_598259.1 | Signal peptide: 1-21 |
| | | Mature: 22-561 |
| | | Ectodomain: 22-338 |
| 7 | Humanized Il1rl2 | Length: 573 aa |
| | Protein | Signal peptide: 1-21 |
| | | Mature: 22-573 |
| | | Ectodomain: 22-337 |

In some embodiments, a rodent disclosed herein comprises a humanized Il1rl2 gene in its genome that includes a nucleotide sequence of an endogenous rodent Il1rl2 gene and a nucleotide sequence of a human IL1RL2 gene. As used herein, “a nucleotide sequence of a gene” includes a genomic sequence, an mRNA or cDNA sequence, in full or in part of the gene. As a non-limiting example, a nucleotide sequence of a human IL1RL2 gene includes a genomic sequence, an mRNA or cDNA sequence, in full or in part of the human IL1RL2 gene. The nucleotide sequence of the endogenous rodent Il1rl2 gene and the nucleotide sequence of the human IL1RL2 gene are operably linked to each other such that the humanized Il1rl2 gene in the rodent genome encodes an Il1rl2 protein, i.e., a protein that has an Il1rl2 protein structure (composed of an ectodomain, a transmembrane domain and a cytoplasmic domain), and that performs Il1rl2 functions (e.g., forms a heterodimer with Interleukin-1 receptor accessory protein (IL-1RacP) and recognizes the three ligands: IL-36α, IL-36β, and IL-36γ).

In some embodiments, a genetically modified rodent contains a humanized Il1rl2 gene in its genome, wherein the humanized Il1rl2 gene encodes a humanized Il1rl2 protein that contains an ectodomain that is substantially identical with the ectodomain of a human IL1RL2 protein. In some embodiments, an ectodomain that is substantially identical with the ectodomain of a human IL1RL2 protein exhibits the same functionality (e.g., ligand binding properties) as the ectodomain of a human IL1RL2 protein. By an ectodomain or polypeptide that is “substantially identical with the ectodomain of a human IL1RL2 protein”, it is meant in some embodiments, a polypeptide that is at least 95%, 98%, 99% or 100% identical in sequence with the ectodomain of a human IL1RL2 protein; in some embodiments, a polypeptide that differs from the ectodomain of a human IL1RL2 protein by not more than 10, 9, 8, 7, 6, 5, 4, 3, 2 or 1 amino acid(s); and in some embodiments, a polypeptide that differs from the ectodomain of a human IL1RL2 protein only at the N- or C-terminal portion of the ectodomain, e.g., by having addition, deletion or substitution of amino acids, but not more than 5, 4, 3, 2 or 1 amino acid at the N- or C-terminal portion of the ectodomain. By “the N- or C-terminal portion of the ectodomain” is meant within 5-10 amino acids from the N- or C-terminus of the ectodomain. In some embodiments, a human IL1RL2 protein has an amino acid sequence substantially identical (e.g., at least 95%, 98%, 99% or 100% identical) with the amino acid sequence as set forth in SEQ ID NO: 2. In specific embodiments, a human IL1RL2 protein comprises the amino acid sequence as set forth in SEQ ID NO: 2, and its ectodomain is composed of amino acids 20-335 of SEQ ID NO: 2. In some embodiments, the humanized Il1rl2 gene encodes a humanized Il1rl2 protein that contains an ectodomain that is substantially identical with the ectodomain of the human IL1RL2 protein as set forth in SEQ ID NO: 2, i.e., with amino acids 20-335 of SEQ ID NO: 2. In some embodiments, the humanized Il1rl2 gene encodes a humanized Il1rl2 protein that contains an ectodomain comprising amino acids 22-337 of SEQ ID NO: 7.

In some embodiments, the humanized Il1rl2 gene encodes a humanized Il1rl2 protein that contains a transmembrane-cytoplasmic sequence (i.e., a sequence that includes both the transmembrane domain and the cytoplasmic domain) that is substantially identical with the transmembrane-cytoplasmic sequence of an endogenous rodent Il1rl2 protein. In some embodiments, a transmembrane-cytoplasmic sequence that is substantially identical with the transmembrane-cytoplasmic sequence of an endogenous rodent Il1rl2 protein exhibits the same functionality (e.g., signal transduction and/or interaction with intracellular molecules) as the transmembrane-cytoplasmic sequence of an endogenous rodent Il1rl2 protein. By a transmembrane-cytoplasmic sequence or polypeptide that is “substantially identical with the transmembrane-cytoplasmic sequence of an endogenous rodent Il1rl2 protein”, it is meant in some embodiments, a polypeptide that is at least 95%, 98%, 99% or 100% identical in sequence with the transmembrane-cytoplasmic sequence of an endogenous rodent Il1rl2 protein; in some embodiments, a polypeptide that differs from the transmembrane-cytoplasmic sequence of an endogenous rodent Il1rl2 protein by not more than 10, 9, 8, 7, 6, 5, 4, 3, 2 or 1 amino acid(s); in some embodiments, a polypeptide that differs from the transmembrane-cytoplasmic sequence of an endogenous rodent Il1rl2 protein only at the N- or C-terminus, e.g., by having addition, deletion or substitution of amino acids, but not more than 5, 4, 3, 2 or 1 amino acid, at the N- or C-terminal portion of the transmembrane-cytoplasmic sequence. By “the N- or C-terminal portion of the transmembrane-cytoplasmic sequence” is meant within 5-10 amino acids from the N-terminus of the transmembrane domain or within 5-10 amino acids from the C-terminus of the cytoplasmic domain. In some embodiments, a humanized Il1rl2 protein contains a transmembrane-cytoplasmic sequence that is substantially identical with the transmembrane-cytoplasmic sequence of a mouse Il1rl2 protein, e.g., a mouse Il1rl2 protein substantially identical (at least 95%, 98%, 99% or 100% identical) with SEQ ID NO: 4, or with the transmembrane-cytoplasmic sequence of a rat Il1rl2 protein, e.g., a rat Il1rl2 protein substantially identical (at least 95%, 98%, 99% or 100% identical) with SEQ ID NO: 6.

In some embodiments, the humanized Il1rl2 gene encodes a humanized Il1rl2 protein that contains a signal peptide that is substantially identical with the signal peptide of an endogenous rodent Il1rl2 protein. By a signal peptide that is “substantially identical with the signal peptide of an endogenous rodent Il1rl2 protein”, it is meant in some embodiments, a polypeptide that is at least 95%, 98%, 99% or 100% identical in sequence with the signal peptide of an endogenous rodent Il1rl2 protein; in some embodiments, a polypeptide that differs from the signal peptide of an endogenous rodent Il1rl2 protein by not more than 3, 2 or 1 amino acid(s); in some embodiments, a polypeptide that differs from the signal peptide of an endogenous rodent Il1rl2 protein only at the N- or C-terminus, e.g., by having addition, deletion or substitution of amino acids, but not more than 3, 2 or 1 amino acid, at the N- or C-terminal portion of the signal peptide. By “the N- or C-terminal portion of the signal peptide” is meant within 5 amino acids from the N- or C-terminus of the signal peptide. In some embodiments, a humanized Il1rl2 protein includes a signal peptide substantially identical with the signal peptide of a mouse Il1rl2 protein, e.g., a mouse Il1rl2 protein substantially identical (at least 95%, 98%, 99% or 100% identical) with SEQ ID NO: 4, or with the signal peptide of a rat Il1rl2 protein, e.g., a rat Il1rl2 protein substantially identical (at least 95%, 98%, 99% or 100% identical) with SEQ ID NO: 6.

In some embodiments, the humanized Il1rl2 gene in the genome of a genetically modified rodent includes a nucleotide sequence of a human IL1RL2 gene (or “a human IL1RL2 nucleotide sequence”) and a nucleotide sequence of an endogenous rodent Il1rl2 gene (or “an endogenous rodent Il1rl2 nucleotide sequence”), wherein the human IL1RL2 nucleotide sequence encodes a polypeptide substantially identical to the ectodomain of the human IL1RL2 protein encoded by the human IL1RL2 gene. Such human IL1RL2 nucleotide sequence is also referred to as encoding substantially the ectodomain of a human IL1RL2 protein. In some embodiments, the human IL1RL2 nucleotide sequence is a genomic fragment of a human IL1RL2 gene. In some embodiments, the human IL1RL2 nucleotide sequence is a genomic fragment comprising exons 3-8 of a human IL1RL2 gene. In some embodiments, the human IL1RL2 nucleotide sequence is a genomic fragment comprising a 3′ portion of intron 2, exons 3-8, and a 5′ portion of intron 8 of a human IL1RL2 gene. In some embodiments, the human IL1RL2 nucleotide sequence is a cDNA sequence.

In some embodiments, the humanized Il1rl2 gene in the genome of a genetically modified rodent includes an endogenous rodent Il1rl2 nucleotide sequence and a human IL1RL2 nucleotide sequence, wherein the endogenous rodent Il1rl2 nucleotide sequence encodes a polypeptide substantially identical to the transmembrane-cytoplasmic sequence of an endogenous rodent Il1rl2 protein. Such rodent Il1rl2 nucleotide sequence is also referred to as encoding substantially the transmembrane-cytoplasmic sequence of the endogenous rodent Il1rl2 protein. In some embodiments, the endogenous rodent Il1rl2 nucleotide sequence present in a humanized Il1rl2 gene encodes the transmembrane-cytoplasmic sequence of the endogenous rodent Il1rl2 protein. In some embodiments, the endogenous rodent Il1rl2 nucleotide sequence present in a humanized Il1rl2 gene comprises the remaining exons downstream of exon 8 in the endogenous rodent Il1rl2 gene. In some embodiments, the endogenous rodent Il1rl2 nucleotide sequence present in a humanized Il1rl2 gene comprises a 3′ portion of intron 8 and the remaining exons downstream of exon 8 in the endogenous rodent Il1rl2 gene.

In some embodiments, the humanized Il1rl2 gene in the genome of a genetically modified rodent includes an endogenous rodent Il1rl2 nucleotide sequence upstream (5′) of a human IL1RL2 nucleotide sequence, wherein the endogenous rodent Il1rl2 nucleotide sequence encodes a polypeptide substantially identical to the signal peptide of the endogenous rodent Il1rl2 protein. Such rodent Il1rl2 nucleotide sequence is also referred to as encoding substantially the signal peptide of an endogenous rodent Il1rl2 protein. In some embodiments, the endogenous rodent Il1rl2 nucleotide sequence encoding a polypeptide substantially identical to the signal peptide of the endogenous rodent Il1rl2 protein comprises exons 1-2 of the endogenous rodent Il1rl2 gene; and in some embodiments, the endogenous rodent Il1rl2 nucleotide sequence includes exons 1-2 and a 5′ portion of intron 2 of an endogenous rodent Il1rl2 gene.

In some embodiments, the humanized Il1rl2 gene is operably linked to endogenous rodent Il1rl2 regulatory sequences, e.g., a 5′ transcriptional regulatory sequence(s) such as promoter and/or enhancers, such as expression of the humanized Il1rl2 gene is under control of the rodent Il1rl2 5′ regulatory sequence(s).

In some embodiments, the humanized Il1rl2 gene is at an endogenous rodent Il1rl2 locus. In some embodiments, the humanized Il1rl2 gene is at a locus other than an endogenous rodent Il1rl2 locus; e.g., as a result of random integration. In some embodiments where the humanized Il1rl2 gene is at a locus other than an endogenous rodent Il1rl2 locus, the rodents are incapable of expressing a rodent Il1rl2 protein, e.g., as a result of inactivation (e.g., deletion in full or in part) of the endogenous rodent Il1rl2 gene.

In some embodiments where a humanized Il1rl2 gene is at an endogenous rodent Il1rl2 locus, the humanized Il1rl2 gene results from a replacement of a nucleotide sequence of an endogenous rodent Il1rl2 gene at the endogenous rodent Il1rl2 locus with a nucleotide sequence of a human IL1RL2 gene.

In some embodiments, the nucleotide sequence of an endogenous rodent Il1rl2 gene at an endogenous rodent Il1rl2 locus that is being replaced is a genomic fragment of an endogenous rodent Il1rl2 gene that encodes substantially the ectodomain of the rodent Il1rl2 protein. In some embodiments, a rodent genomic fragment being replaced comprises exons 3-8 of an endogenous rodent Il1rl2 gene.

In some embodiments, the nucleotide sequence of a human IL1RL2 gene that replaces a genomic fragment of a rodent Il1rl2 gene at an endogenous rodent Il1rl2 locus is a cDNA sequence. In some embodiments, the human IL1RL2 nucleotide sequence that replaces a genomic fragment of a rodent Il1rl2 gene at an endogenous rodent Il1rl2 locus is a genomic fragment of a human IL1RL2 gene. In some embodiments, a genomic fragment of a human IL1RL2 gene that replaces a genomic fragment of a rodent Il1rl2 gene at an endogenous rodent Il1rl2 locus includes exons, in full or in part, of a human IL1RL2 gene, that encode substantially the ectodomain of the human IL1RL2 protein. In some embodiments, the human genomic fragment comprises exons 3-8 of a human IL1RL2 gene.

In some embodiments, the genomic sequence of an endogenous rodent Il1rl2 gene that remains at an endogenous rodent Il1rl2 locus after the humanization replacement and is operably linked to the inserted human IL1RL2 nucleotide sequence encodes substantially the transmembrane-cytoplasmic sequence of the endogenous rodent Il1rl2 protein. In some embodiments, the genomic sequence of an endogenous rodent Il1rl2 gene that remains at an endogenous rodent Il1rl2 locus after the humanization replacement includes the exons downstream of exon 8 of the endogenous rodent Il1rl2 gene.

In some embodiments, the genomic sequence of an endogenous rodent Il1rl2 gene that remains at an endogenous rodent Il1rl2 locus after the humanization replacement and is operably linked to the inserted human IL1RL2 nucleotide sequence encodes substantially the signal peptide of the endogenous rodent Il1rl2 protein. In some embodiments, the genomic sequence of an endogenous rodent Il1rl2 gene that remains at an endogenous rodent Il1rl2 locus after the humanization replacement includes exons 1-2 of the endogenous rodent Il1rl2 gene.

In some embodiments, in circumstances where an endogenous rodent Il1rl2 protein and a human IL1RL2 protein share common amino acids near the junction between the transmembrane domain and the ectodomain, it may not be necessary to insert a human IL1RL2 nucleotide sequence that encodes precisely the ectodomain of the human IL1RL2 protein. It is possible to insert a slightly longer or shorter nucleotide sequence of a human IL1RL2 gene, which encodes substantially the ectodomain of the human IL1RL2 protein, in operable linkage to a genomic sequence of an endogenous rodent Il1rl2 gene that encodes substantially the transmembrane domain (and the cytoplasmic domain) of the endogenous rodent Il1rl2 protein, such that the humanized Il1rl2 protein encoded by the resulting humanized Il1rl2 gene includes an ectodomain that is identical with the ectodomain of the human IL1RL2 protein and a transmembrane domain that is identical with the transmembrane domain of the endogenous rodent Il1rl2 protein.

In some embodiments, a genomic fragment comprising exons 3-8 of an endogenous rodent Il1rl2 gene at an endogenous rodent Il1rl2 locus has been replaced with a genomic fragment comprising exons 3-8 of a human IL1RL2 gene. As a result, a humanized Il1rl2 gene is formed at the endogenous rodent Il1rl2 locus and comprises exons 1-2 of the endogenous rodent Il1rl2 gene, exons 3-8 of the human IL1RL2 gene, and the remaining exons downstream of exon 8 of the endogenous rodent Il1rl2 gene.

In some embodiments, a rodent provided herein is heterozygous for a humanized Il1rl2 gene in its genome. In some embodiments, a rodent provided herein is homozygous for a humanized Il1rl2 gene in its genome.

In some embodiments, a humanized Il1rl2 gene results in an expression of the encoded humanized Il1rl2 protein in a rodent. In some embodiments, a humanized Il1rl2 protein is expressed in a pattern comparable with, or substantially the same as, a counterpart rodent Il1rl2 protein in a control rodent (e.g., a rodent without the humanized Il1rl2 gene). In some embodiments, a humanized Il1rl2 protein is expressed at a level comparable with, or substantially the same as, a counterpart rodent Il1rl2 protein in a control rodent (e.g., a rodent without the humanized Il1rl2 gene). In some embodiments, a humanized Il1rl2 protein is expressed and detected at the cell surface, e.g., but not limited to, surface of cells such as keratinocytes, monocytes, macrophages, neutrophils, bronchial and intestinal epithelial cells among others. In the context of comparing a humanized gene or protein in a humanized rodent with an endogenous rodent gene or protein in a control rodent, the term “comparable” means that the molecules or levels being compared may not be identical to one another but are sufficiently similar to permit comparison there between so that conclusions may reasonably be drawn based on differences or similarities observed. In some embodiments, the term “substantially the same”, in referring to expression levels, include levels being compared that are not different from one another by more than 20%, 19%, 18%, 17%, 16%, 15%, 14%, 13%, 12%, 11%, 10%, 9%, 8%, 7%, 6%, 5%, 4%, 3%, 2%, or 1%.

In some embodiments, rodents disclosed herein are incapable of expressing a rodent Il1rl2 protein, e.g., as a result of inactivation (e.g., deletion in full or in part) or replacement (in full or in part) of the endogenous rodent Il1rl2 gene.

### IL-36 Ligands Humanization

The rodents disclosed herein also comprise in the germline genes encoding human IL-36α, β, and γ ligands.

TABLE 2

| SEQ | | |
|---|---|---|
| ID NO | Description | Features |
| 8 | Homo sapiens IL1F6 (IL-36α) | Length: 888 bp |
| | mRNA, NM_014440.2 | CDS: 412-888 |
| 9 | Homo sapiens IL1F6 (IL-36α) | Length: 158aa |
| | protein, NP_055255.1 | Mature: 6-158 |
| 10 | Mus musculus Il1f6 (Il-36α) | Length: 883 bp |
| | mRNA, NM_019450.3 | CDS: 162-644 |
| 11 | Mus musculus Il1f6 (Il-36α) | Length: 160 aa |
| | Protein NP_062323.1 | Mature: 8-160 |
| 12 | Rattus norvegicus Il1f6 (Il-36α) | Length: 869 bp |
| | mRNA, NM_001106554.1 | CDS: 162-644 |
| 13 | Rattus norvegicus Il1f6 (Il-36α) | Length: 160 aa |
| | Protein, NP_001100024.1 | |

In some embodiments, a rodent disclosed herein has a genome comprising a human IL-36α gene that encodes a human IL-36α protein. References to a “human IL-36α gene” include a human genomic DNA that encodes a human IL-36α protein and comprises human IL-36α promoter. A human IL-36α protein can be the mature form or the precursor form of a human IL-36α protein. In some embodiments, a human IL-36α protein comprises the amino acid sequence of SEQ ID NO: 9.

In some embodiments, a human IL-36α gene is at an endogenous rodent Il-36α locus. In some embodiments, a human IL-36α gene is at a locus other than an endogenous rodent Il-36α locus; e.g., as a result of random integration. In some embodiments where the human IL-36α gene is at a locus other than an endogenous rodent Il-36α locus, the rodents are incapable of expressing a rodent Il-36α protein, e.g., as a result of inactivation (e.g., deletion in full or in part) of the endogenous rodent Il-36α gene.

In some embodiments, a human IL-36α gene replaces an endogenous rodent Il-36α gene at an endogenous rodent Il-36α locus.

In some embodiments, a rodent provided herein is heterozygous for a human IL-36α gene in its genome. In other embodiments, a rodent provided herein is homozygous for a human IL-36α gene in its genome.

In some embodiments, a human IL-36α gene results in an expression of the encoded human IL-36α protein in a rodent, e.g., in serum, at mucosal sites, such as skin, intestinal epithelium, lungs, and in various types of cells of immune system (e.g., monocytes, macrophages, T cells, dendritic cells). In some embodiments, the human IL-36α protein is expressed in a pattern comparable with, or substantially the same as, a counterpart rodent Il-36α protein in a control rodent (e.g., a rodent without the human IL-36α gene). In some embodiments, the human IL-36α protein is expressed at a level comparable with, or substantially the same as, a counterpart rodent Il-36α protein in a control rodent (e.g., a rodent without the human IL-36α gene), e.g., in serum, at mucosal sites (e.g., such as skin, intestinal epithelium, lungs), and/or in immune cells (e.g., monocytes, macrophage, T cells, dendritic cells).

In some embodiments, rodents disclosed herein are incapable of expressing a rodent Il-36α protein, e.g., as a result of inactivation (e.g., deletion in full or in part) or replacement (in full or in part) of the endogenous rodent Il-36α gene.

TABLE 3

| SEQ | | |
|---|---|---|
| ID NO | Description | Features |
| 14 | Homo sapiens IL1F8 (IL-36β), | Length: 1186 bp |
| | transcript variant 1, mRNA, | CDS: 109-603 |
| | NM_014438.4 | |
| 15 | Homo sapiens IL1F8 (IL-36β) | Length: 164 aa |
| | protein, NP_055253.2 | |
| 16 | Mus musculus Il1f8 (Il-36β) | Length: 790 bp |
| | mRNA, NM_027163.4 | CDS: 66-617 |
| 17 | Mus musculus Il1f8 (Il-36β) | Length: 183 aa |
| | Protein NP_081439.1 | |
| 18 | Rattus norvegicus Il1f8 (Il-36β) | Length: 587 bp |
| | mRNA, NM_001108570.1 | CDS: 92-415 |
| 19 | Rattus norvegicus Il1f8 (Il-36β) | Length: 179 aa |
| | Protein, XP_006233676 | |

In some embodiments, the rodents disclosed herein have a genome comprising a human IL-36β gene that encodes a human IL-36β protein. References to a “human IL-36β gene” include a human genomic DNA that encodes a human IL-36β protein and comprises human IL-36β promoter. A human IL-36β protein can be the mature form or the precursor form of a human IL-36β protein. In some embodiments, a human IL-36β protein comprises the amino acid sequence of SEQ ID NO: 15.

In some embodiments, a human IL-36β gene is at an endogenous rodent Il-36β locus. In some embodiments, a human IL-366 gene is at a locus other than an endogenous rodent Il-36β locus; e.g., as a result of random integration. In some embodiments where the human IL-36β gene is at a locus other than an endogenous rodent Il-36β locus, the rodents are incapable of expressing a rodent Il-36β protein, e.g., as a result of inactivation (e.g., deletion in full or in part) of the endogenous rodent Il-36β gene.

In some embodiments, a human IL-36β gene replaces an endogenous rodent Il-36β gene at an endogenous rodent II-36β locus.

In some embodiments, a rodent provided herein is heterozygous for a human IL-36β gene in its genome. In other embodiments, a rodent provided herein is homozygous for a human IL-36β gene in its genome.

In some embodiments, a human IL-36β gene results in an expression of the encoded human IL-36β protein in a rodent, e.g., in serum, at mucosal sites (e.g., such as skin, intestinal epithelium, lungs), and/or in immune cells (e.g., monocytes, macrophage, T cells, dendritic cells). In some embodiments, the human IL-36β protein is expressed in a pattern comparable with, or substantially the same as, a counterpart rodent Il-36β protein in a control rodent (e.g., a rodent without the human IL-36β gene). In some embodiments, the human IL-36β protein is expressed at a level comparable with, or substantially the same as, a counterpart rodent Il-36β protein in a control rodent (e.g., a rodent without the human IL-36β gene), e.g., in serum, at mucosal sites, such as skin, intestinal epithelium, lungs, and in various types of cells of immune system (e.g., monocyte, macrophages, T cells, dendritic cells).

In some embodiments, rodents disclosed herein are incapable of expressing a rodent I Il-36β protein, e.g., as a result of inactivation (e.g., deletion in full or in part) or replacement (in full or in part) of the endogenous rodent Il-36β gene.

TABLE 4

| SEQ | | |
|---|---|---|
| ID NO | Description | Features |
| 20 | Homo sapiens IL1F9 (IL-36γ), | Length: 1212 bp |
| | transcript variant 1, mRNA, | CDS: 80-589 |
| | NM_019618.3 | |
| 21 | Homo sapiens IL1F9 (IL-36γ) | Length: 169 aa |
| | protein, NP_062564.1 | |
| 22 | Mus musculus Il1f9 (Il-36γ) | Length: 1647 bp |
| | mRNA, NM_153511.3 | CDS: 295-876 |
| 23 | Mus musculus Il1f9 (Il-36γ) | Length: 164 aa |
| | Protein, Q8R460.1 | |
| 24 | Rattus norvegicus Il1f9 (Il-36γ) | Length: 1395 bp |
| | mRNA, NM_001113790.1 | CDS: 166-663 |
| 25 | Rattus norvegicus Il1f9 (Il-36γ) | Length: 165 aa |
| | Protein, NP_001107262.1 | |

In some embodiments, this invention provides a rodent whose genome contains a human IL-36γ gene that encodes a human IL-36γ protein. References to a “human IL-36γ gene” include a human genomic DNA that encodes a human IL-36γ protein and comprises human IL-36γ promoter. In some embodiments, a human IL-36γ protein can be the mature form or the precursor form of a human IL-36γ protein. In some embodiments, a human IL-36γ protein comprises the amino acid sequence of SEQ ID NO: 21.

In some embodiments, a human IL-36γ gene is at an endogenous rodent Il-36γ locus. In some embodiments, a human IL-36γ gene is at a locus other than an endogenous rodent Il-36γ locus; e.g., as a result of random integration. In some embodiments where the human IL-36γ gene is at a locus other than an endogenous rodent Il-36γ locus, the rodents are incapable of expressing a rodent Il-36γ protein, e.g., as a result of inactivation (e.g., deletion in full or in part) of the endogenous rodent Il-36γ gene.

In some embodiments, a human IL-36γ gene replaces an endogenous rodent Il-36γ gene at an endogenous rodent Il-36γ locus.

In some embodiments, a rodent provided herein is heterozygous for a human IL-36γ gene in its genome. In other embodiments, a rodent provided herein is homozygous for a human IL-36γ gene in its genome.

In some embodiments, a human IL-36γ gene results in an expression of the encoded human IL-36γ protein (e.g., a protein identical with a human IL-36γ protein) in a rodent, e.g., in serum, at mucosal sites, such as skin, intestinal epithelium, lungs, and in various types of cells of immune system (e.g., monocyte, macrophages, T cells, dendritic cells). In some embodiments, the human IL-36γ protein is expressed in a pattern comparable with, or substantially the same as, a counterpart rodent Il-36γ protein in a control rodent (e.g., a rodent without the human IL-36γ gene). In some embodiments, the human IL-36γ protein is expressed at a level comparable with, or substantially the same as, a counterpart rodent Il-36γ protein in a control rodent (e.g., a rodent without the human IL-36γ gene), e.g., in serum, at mucosal sites (e.g., such as skin, intestinal epithelium, lungs), and/or in immune cells (e.g., monocytes, macrophage, T cells, dendritic cells).

In some embodiments, rodents disclosed herein are incapable of expressing a rodent Il-36γ protein, e.g., as a result of inactivation (e.g., deletion in full or in part) or replacement (in full or in part) of the endogenous rodent Il-36γ gene.

In some embodiments, rodents are provided whose genome comprise a replacement of a contiguous genomic fragment encompassing the coding sequences for all three Il-36 ligands at an endogenous locus with a contiguous nucleic acid comprising coding sequences for three ligands that are substantially identical with a human IL-36α, β, and γ, respectively. In some embodiments, the resulting locus comprises, from 5′ to 3′, (i) a human IL-36β gene, (ii) a human IL-36γ gene, and (iii) the reverse strand of a human IL-36α gene.

### Phenotypes of Quadruple Humanized Rodents

The genetically modified rodents disclosed herein do not develop any spontaneous diseases at steady state; however, these rodents do display shortened colons and increased expression of proinflammatory mediators in the skin as compared to age-matched control rodents without the humanization, at both steady state and disease state (e.g., after DSS or IMQ treatment). In some embodiments, a genetically modified rodent displays a colon length that is at least 10%, 15%, 20%, 25%, 30%, 35%, 40%, 45%, or 50% shorter than a control rodent at steady state or disease state.

In some embodiments, a genetically modified rodent displays a colon length that is 10% to 15% (+/−5%) shorter than a control rodent at steady state. In some embodiments, a genetically modified rodent displays a colon length that is 15% to 20% (+/−5%) shorter than a control rodent at steady state. In some embodiments, a genetically modified rodent displays a colon length that is 10% to 20% (+/−5%) shorter than a control rodent at steady state.

In other embodiments, a genetically modified rodent displays a colon length that is 20% to 40% (+/−5%) shorter than a control rodent at disease state (e.g., after DSS or oxazolone treatment). In other embodiments, a genetically modified rodent displays a colon length that is 30% to 40% (+/−5%) shorter than a control rodent at disease state (e.g., after DSS or oxazolone treatment). In other embodiments, a genetically modified rodent displays a colon length that is 20% to 30% (+/−5%) shorter than a control rodent at disease state (e.g., after DSS or oxazolone treatment). In other embodiments, a genetically modified rodent displays a colon length that is 25% to 35% (+/−5%) shorter than a control rodent at disease state (e.g., after DSS or oxazolone treatment).

Although the genetically modified rodents disclosed herein do not develop any spontaneous diseases at steady state, they have been shown to display enhanced skin and intestinal inflammation in experimentally induced skin and intestinal inflammation models (e.g., preclinical models of psoriasis and IBD, respectively).

In some embodiments, DSS is used to induce chronic colitis. In some embodiments, DSS is administered to rodents through drinking water at least 0.5%, at least 1%, at least 1.5%, at least 2.5%. In some embodiments, DSS is administered to rodents through drinking water at not more than 10%, 9%, 8%, 7%, 6%, or 5%. In some embodiments, drinking water containing DSS at 1.5%-3% is given to rodents. In some embodiments, drinking water containing DSS at 0.5%-3% is given to rodents. In some embodiments, drinking water containing DSS at 1%-3% is given to rodents. In some embodiments, drinking water containing DSS at 2%-3% is given to rodents. In some embodiments, drinking water containing DSS at 2.5%-3% is given to rodents. In some embodiments, drinking water containing DSS at 0.5%-2.5% is given to rodents. In some embodiments, drinking water containing DSS at 0.5%-2% is given to rodents. In some embodiments, drinking water containing DSS at 0.5%-1.5% is given to rodents. In some embodiments, drinking water containing DSS at 0.5%-1% is given to rodents. In some embodiments, drinking water containing DSS at 1%-2.5% is given to rodents. In some embodiments, drinking water containing DSS at 1.5%-2% is given to rodents.

In some embodiments, administration of DSS can be performed for a period of 5, 6, 7, 8, 9, 10, 11, 12, 13, 14, 15, 16, 17, 18, 19, 20 or more days, and can be continuous or interrupted by days without a DSS administration. In some embodiments, rodents are provided with drinking water containing 2.5% DSS for 7 days, then 1.5% DSS for 5 days, followed by distilled water until 27-30 days before analysis. In some embodiments, rodents are provided with drinking water containing 3% DSS for 7 days, then 2% DSS for 13 days, followed by distilled water until 27-30 days before analysis. In some embodiments, DSS is not given for the total period. In some embodiments, rodents are provided with drinking water containing 2.5% DSS for 7 days, followed by 11 days of distilled water, then 1.5% DSS for 4 days, followed by 5 days of distilled water—for a total of 27 days before analysis. In some embodiments, rodents are provided with drinking water containing 3% DSS for 7 days, followed by 13 days of water, then 2% DSS for 4 days, followed by 6 days of distilled water until 30 days before analysis.

In some embodiments, oxazolone is used to induce colitis. In some embodiments, oxazolone is given to rodents intrarectally to induce colitis. In some embodiments, oxazolone is given to rodents intrarectally in three administrations to induce colitis. In some embodiments, oxazolone is applied to rodents topically for pre-sensitization prior to intrarectal administration. In some embodiments, oxazolone is given to a rodent for pre-sensitization by topical application of a solution of oxazolone (e.g., 3% solution of oxazolone dissolved in 100% ethanol) on the shaved skin, followed by a three intrarectal administrations of a solution of oxazolone (e.g., 1.0-2.0% oxazolone dissolved in 50% ethanol) . . . . In some embodiments, the pre-sensitization employs a solution of 1.5%, 2.0%, 2.5%, 3.0%, 3.5%, or 4.0% oxazolone dissolved in 100% ethanol. In some embodiments, the intrarectal administrations employ a solution of 1.0%, 1.1%, 1.2%, 1.3%, 1.5%, 1.6%, 1.7%, 1.8%, 1.9% or 2.0% of oxazolone dissolved in 50% ethanol. In some embodiments, the intrarectal administration is performed several days, e.g., 3, 4, 5, 6 or 7 days, after the pre-sensitization. In some embodiments, oxazolone is given to a rodent by topical application on the skin in a solution of 3.0% oxazolone dissolved in 100% ethanol, followed by three intrarectal administrations of a solution of 1.0-2.0% oxazolone dissolved in 50% ethanol on days 5, 6 and 7.

In some embodiments, the extent of colitis is evaluated by scoring the following features: inflammation (severity and extent), epithelial changes (erosion/ulcer), changes in the crypts (crypt loss, cryptitis/crypt abscess, regeneration/hyperplasia, goblet cell loss), submucosal edema and percentage of tissue area with pathology relative to the total tissue area on the slide. A 0-4 scoring scale is used: 0-O-within normal limits, 1-minimal, 2-mild, 3-moderate and 4-severe. A total pathology score is calculated for each rodent animal by adding the individual histopathological feature scores. In some embodiments, colitis is evaluated by measuring the level of Lipocalin-2 (Lcn2) in fecal samples. In some embodiments, colitis is measured by measuring the level of Myeloperoxidase (MPO) activity in colon homogenates. In some embodiments, colitis is evaluated by measuring the levels of inflammatory cytokines in colon homogenates.

In some embodiments, the genetically modified rodents display an increased intestinal pathology score, e.g., by at least 50%, 60%, 70%, 80%, 90%, 100%, 200%, 300%, 400% or more, as compared to wild type control rodents that have undergone the same DSS administration. In some embodiments, the genetically modified rodents display an increased intestinal pathology score by, e.g., 50%-400%, 50%-300%, 50%-200%, 50%-100%, 100%-400%, 100%-300%, 100-200%, or 200%-400%, as compared to wild type control rodents that have undergone the same DSS administration. In some embodiments, the genetically modified rodents exhibit an increased level of Myeloperoxidase (“MPO”) in colon homogenates, e.g., by at least 50%, 60%, 70%, 80%, 90%, 100%, 200%, 300%, 400%, or more, as compared to wild type control rodents that have undergone the same DSS administration. In some embodiments, the genetically modified rodents display an increased level of MPO in colon homogenates by, e.g., 50%-400%, 50%-300%, 50%-200%, 50%-100%, 100%-400%, 100%-300%, 100-200%, or 200%-400%, as compared to wild type control rodents that have undergone the same DSS administration. In some embodiments, the genetically modified rodents exhibit an increased level of fecal Lcn, e.g., by at least 10%, 20%, 30%, 40%, 50%, 60%, 70%, 80%, 90%, 100%, 200%, 300%, 400%, or more, as compared to wild type control rodents that have undergone the same DSS administration. In some embodiments, the genetically modified rodents exhibit an increased mRNA expression and/or protein level of one or more proinflammatory mediators (e.g., KC-GRO, IL-6, IL-1β, TNFα, IL-21, IL-12p40, IL-17f, IL-17a, and IL-17c) in colon homogenates, e.g., by at least 20%, 30%, 40%, 50%, 60%, 70%, 80%, 90%, 100%, 200%, 300%, 400%, 500%, 600%, 700%, 800%, 900% or more, as compared to wild type control rodents that have undergone the same DSS administration. In some embodiments, the genetically modified rodents exhibit an increased mRNA expression and/or protein level of one or more proinflammatory mediators in colon homogenates by, e.g., 20%-900%, 20%-800%, 20%-700%, 20%-600%, 20%-500%, 20%-400%, 20%-300%, 20%-200%, or 20%-100%. In some embodiments, the genetically modified rodents exhibit an increased mRNA expression and/or protein level of one or more proinflammatory mediators in colon homogenates by, e.g., 30%-900%, 30%-800%, 30%-700%, 30%-600%, 30%-500%, 30%-400%, 30%-300%, 30%-200%, or 30%-100%. In some embodiments, the genetically modified rodents exhibit an increased mRNA expression and/or protein level of one or more proinflammatory mediators in colon homogenates by, e.g., 40%-900%, 40%-800%, 40%-700%, 40%-600%, 40%-500%, 40%-400%, 40%-300%, 40%-200%, or 40%-100%. In some embodiments, the genetically modified rodents exhibit an increased mRNA expression and/or protein level of one or more proinflammatory mediators in colon homogenates by, e.g., 50%-900%, 50%-800%, 50%-700%, 50%-600%, 50%-500%, 50%-400%, 50%-300%, 50%-200%, or 50%-100%.

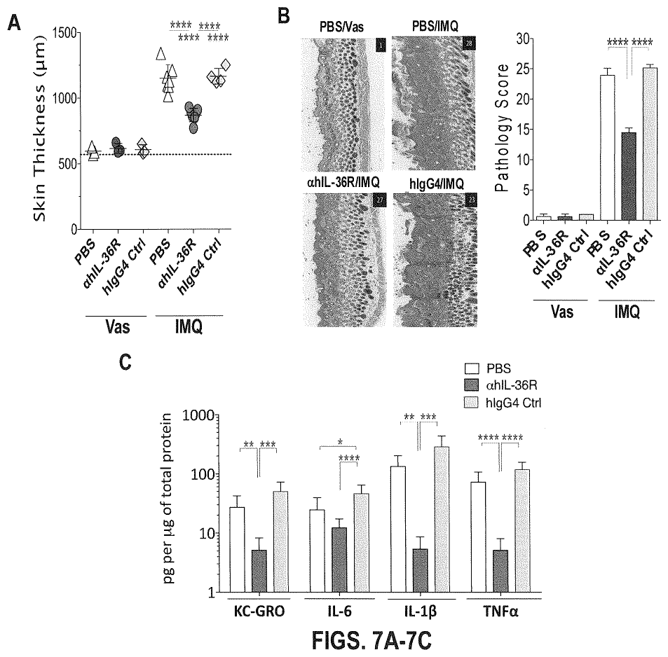

In some embodiments, IMQ is applied topically to the skin of a rodent to induce skin inflammation. In some embodiments, IMQ is provided in a carrier suitable for topical application, e.g., a cream, a gel, including commercially available IMQ creams (e.g., such as those from Aldara). In some embodiments, to induce skin inflammation, IMQ is applied to the rodent skin daily at a daily dose of 1 to 5 mg, 2 to 4 mg, or 3-3.5 mg, for a period of 2, 3, 4, 5, 6, 7, 8, 9, 10 days or longer. In some embodiments, a daily topical application at a daily dosage of about 3.125 mg for 4 days is suitable for inducing acute skin inflammation, and a daily topical application at a daily dosage of about 3.125 mg for 9 days is suitable for inducing chronic skin inflammation. In some embodiments, IMQ is applied topically for multiple rounds (e.g., 2, 3 or 4 rounds), with 4-5 consecutive days of IMQ application followed by 2 days of no IMQ application for each round, before analysis or assay is performed. In a specific embodiment, IMQ is applied topically for two rounds, with 5 consecutive days of IMQ application followed by 2 days of no IMQ application for the first round, then 4 consecutive days of IMQ application for the second round, before analysis (see, e.g., FIGS. 7A-7C). In some embodiments, the severity of inflammation can be evaluated by (i) using an adapted version of the clinical Psoriasis Area and Severity Index based on measuring erythema, scaling and thickening of the skin; (ii) performing histopathological analysis of skin tissues, e.g., to evaluate the presence of parakeratosis, orthokeratosis, Munro's microabscess, acanthosis, epidermal ulceration, inflammation in the dermis and hypodermis, blood vessel congestion in the dermis and hypodermis, follicular hyperkeratosis and epithelial hyperplasia, and to determine a total pathology score; (iii) measuring mRNA expression and/or protein levels of proinflammatory mediators in the skin homogenates, including e.g., mRNA expression and/or protein levels of Cxcl1, IL-17f, IL-17a, IL-23a, S100A8 and Defb4, among others; and (iv) a combination of (i)-(iii).

In some embodiments, the genetically modified rodents display an increased skin pathology score, e.g., by at least 20%, 30%, 40%, 50%, 60%, 70%, 80%, 90%, 100%, 200% or more, as compared to wild type control rodents that have undergone the same IMQ administration. In some embodiments, the genetically modified rodents exhibit an increased mRNA expression and/or protein level of one or more proinflammatory mediators (e.g., but not limited to, Cxcl1, IL-17f, IL-17a, IL-23a, S100A8 and Defb4) in skin homogenates, e.g., by at least 20%, 30%, 40%, 50%, 60%, 70%, 80%, 90%, 100%, 200%, 300%, or more, as compared to wild type control rodents that have undergone the same IMQ administration. In some embodiments, the genetically modified rodents at steady state exhibit an increased mRNA expression and/or protein level of one or more proinflammatory mediators (e.g., but not limited to, Cxcl1, IL-17f, IL-17a, IL-23a, S100A8 and Defb4) in skin homogenates, e.g., by at least 20%, 30%, 40%, 50%, 60%, 70%, 80%, 90%, 100%, 200%, 300%, or more, as compared to wild type control rodents at steady state.

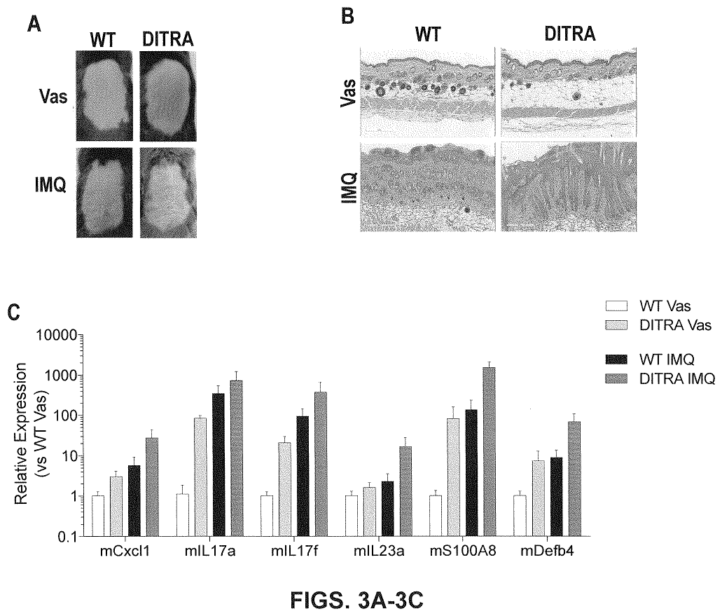

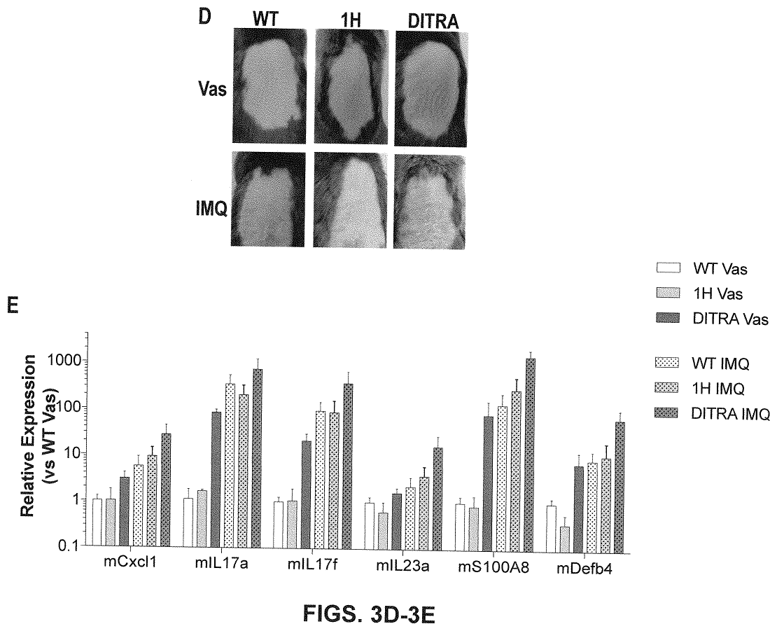

For IMQ induced-skin inflammation, single Il1rl2 humanized mice exhibited phenotypes similar to a wild type rodent, as reflected in histopathology and RNA expression of pro-inflammatory molecules in the skin (see FIGS. 3D and 3E). On the other hand, DITRA-like mice (i.e., quadruple-humanized mice comprising humanized Il1rl2 and human IL1F6, IL1F8, and IL1F9) developed increased skin inflammation compared to both WT and 1H mice after IMQ application (see FIGS. 3D and 3E).

### Tissues and Cells of Genetically Modified Rodents

In some embodiments, disclosed herein is an isolated cell or tissue from a rodent animal as described herein. In some embodiments, a cell is selected from a dendritic cell, lymphocyte (e.g., a B or T cell), macrophage and a monocyte. In some embodiments, a tissue is selected from adipose, bladder, brain, breast, bone marrow, eye, heart, intestine, kidney, liver, lung, lymph node, muscle, pancreas, plasma, serum, skin, spleen, stomach, thymus, testis, ovum, and a combination thereof. In some embodiments, the isolated cell or tissue comprises in its genome the quadruple humanization features described herein.

### Compositions and Methods for Making Quadruple Humanized Rodents

Further aspects of this disclosure are directed to methods for making a genetically modified rodent described above, as well as nucleic acid vectors and rodent embryonic stem cells suitable for use in making a genetically modified rodent.

A quadruple-humanized rodent, i.e., a rodent comprising humanized Il1rl2 and human IL1F6, IL1F8, and IL1F9 genes, can be generated by making a single Il1rl2 humanized rodent strain, and a Il1f6, Il1f8, and Il1f9 triple human rodent strain, followed by breeding the single-humanized and triple-human strains together to obtain a quadruple-humanized rodent. The term “breeding” or “crossing”, as used herein in reference to rodents, refers to the mating of the rodents to produce offspring. Those skilled in the art would understand that more than one cross may be needed in order to achieve homozygosity.

In some embodiments, disclosed herein is a targeting vector (or nucleic acid construct) comprises a human nucleotide sequence desired to be integrated into a rodent locus. In some embodiments, the human nucleotide sequence can be a nucleotide sequence of a human IL1RL2 gene that encodes an ectodomain substantially identical to a human IL1RL2 ectodomain, e.g., a nucleotide sequence comprising exons 3-8 of a human IL1RL2 gene. In some embodiments, the human nucleotide sequence can be a nucleotide sequence encompassing the coding sequence for human IL-36α, the coding sequence for human Il-36β and the complement strand of the coding sequence of a human IL-36γ. The targeting vector also includes 5′ and 3′ rodent sequences flanking the human nucleotide sequence to be integrated, also known as homology arms, that mediate homologous recombination and integration of the human nucleotide sequence into the target rodent locus (e.g., the Il1rl2 locus, or the locus where the rodent Il1f6, Il1f8, and Il1f9 genes are located). In some embodiments, the 5′ and 3′ flanking rodent sequences are the nucleotide sequences that flank the corresponding rodent nucleotide sequence at the target rodent locus that is to be replaced by the human nucleotide sequence. For example, in embodiments where a rodent ectodomain-encoding nucleotide sequence (e.g., exons 3-8 of a rodent Il1rl2 gene) is replaced with a human ectodomain-encoding sequence (e.g., exons 3-8 of a human IL1RL2 gene), the 5′ flanking sequence can include exons 1-2 of the rodent Il1rl2 gene, and the 3′ flanking sequence can include the remaining exons downstream of exon 8 of the rodent Il1rl2 gene. In some embodiments where the rodent nucleotide sequence coding for all three IL-36 ligands is to be replaced with a human nucleotide sequence, the 5′ flanking sequence can include a rodent nucleotide sequence upstream of the coding sequence of the Il1f6 gene, and the 3′ flanking sequence can include a rodent nucleotide sequence upstream of the coding sequence of the Il1f9 gene.

In some embodiments, a targeting vector comprises a selection marker gene. In some embodiments, a targeting vector comprises one or more site-specific recombination sites. In some embodiments, a targeting vector comprises a selection marker gene, flanked by site-specific recombination sites, such that the selection marker gene can be deleted as a result of recombination between the sites.

In exemplary embodiments, a bacterial artificial chromosome (BAC) clone carrying a rodent genomic fragment can be modified using bacterial homologous recombination and VELOCIGENE® technology (see, e.g., U.S. Pat. No. 6,586,251 and Valenzuela et al. (2003) Nature Biotech. 21(6):652-659; which are incorporated herein by reference in their entireties). As a result, a rodent genomic sequence is deleted from the original BAC clone, and a human nucleotide sequence is inserted, resulting in a modified BAC clone carrying a human nucleotide sequence, flanked with 5′ and 3′ rodent homology arms. In some embodiments, human nucleotide sequence can be a cDNA sequence or a human genomic DNA encoding (i) human IL1RL2 in whole or in part (e.g., the ectodomain of a human IL1RL2 protein), or (ii) all three of human IL1F6, IL1F8, and IL1F9. The modified BAC clone, once linearized, can be introduced into rodent embryonic stem (ES) cells.

In some embodiments, the present invention provides use of a targeting vector as described herein to make a modified rodent embryonic stem (ES) cell. For example, a targeting vector can be introduced into a rodent ES cell by, e.g., electroporation. Both mouse ES cells and rat ES cells have been described in the art. See, e.g., U.S. Pat. Nos. 7,576,259, 7,659,442, 7,294,754, and US 2008-0078000 A1 (all of which are incorporated herein by reference in their entireties) that describe mouse ES cells and the VELOCIMOUSE® method for making a genetically modified mouse; US 2014/0235933 A1 (Regeneron Pharmaceuticals, Inc.), US 2014/0310828 A1 (Regeneron Pharmaceuticals, Inc.), Tong et al. (2010) Nature 467:211-215, and Tong et al. (2011) Nat Protoc. 6 (6): doi: 10.1038/nprot.2011.338 (all of which are incorporated herein by reference in their entireties) that describe rat ES cells and methods for making a genetically modified rat, which can be used to make a modified rodent embryo, which in turn can be used to make a rodent animal.

In some embodiments, ES cells having a human nucleotide sequence integrated in the genome can be selected. In some embodiments, ES cells are selected based on loss of rodent allele and/or gain of human allele assays. In some embodiments, selected ES cells are then used as donor ES cells for injection into a pre-morula stage embryo (e.g., 8-cell stage embryo) by using the VELOCIMOUSE® method (see, e.g., U.S. Pat. Nos. 7,576,259, 7,659,442, 7,294,754, and US 2008-0078000 A1, all of which are incorporated by reference in their entireties), or methods described in US 2014/0235933 A1 and US 2014/0310828 A1, which are both incorporated by reference in their entireties. In some embodiments, the embryo comprising the donor ES cells is incubated until blastocyst stage and then implanted into a surrogate mother to produce an F0 rodent fully derived from the donor ES cells. Rodent pups bearing a human nucleotide sequence can be identified by genotyping of DNA isolated from tail snips using loss of rodent allele and/or gain of human allele assays.

In some embodiments, rodents heterozygous for a humanized gene can be crossed to generate homozygous rodents.

### Methods Employing the Quadruple Humanized Rodents

Rodents disclosed herein provide a useful in vivo system and source of biological materials for identifying and testing compounds useful for treating a disease or condition associated with deregulated IL-36 signaling.

By “disease associated with deregulated IL-36 signaling” it is meant a disease in which abnormal IL-36 signaling is manifested, which could cause directly or indirectly the disease, or exacerbate symptoms of the disease. Non-limiting examples of diseases associated with deregulated IL-36 signaling include generalized pustular psoriasis (GPP or DITRA) (Marrakchi S. et al., N Engl J. Med. 365:620-628 (2011) and Onoufriadis A. et al., Am J. Hum Genet 89:432-437 (2011), which are herein incorporated by reference in their entireties), palmoplantar pustular psoriasis (PPPP) (Bissonnette R. et al., PLoS One 11: e0155215 (2016), which is herein incorporated by reference in its entirety), inflammatory bowel disease (IBD) (Medina-Contreras et al., J Immunol 196:34-38 (2016; Nishida A. et al., Inflamm Bowel Dis 22:303-314 (2016) and Russell S E et al., Mucosal Immunol. 9:1193-1204 (2016) which are herein incorporated by reference in their entireties), rheumatoid and psoriatic arthritis (Frey S. et al., Ann Rheum Dis 72:1569-1574 (2013), which is herein incorporated by reference in its entirety), asthma, chronic obstructive pulmonary disease (Chen H. et al., J. Proteomics 75:2835-2843 (2012), which is herein incorporated by reference in its entirety), chronic kidney disease (Shaik Y. et al., Int J Immunopathol Pharmacol 26:27-36 (2013) which is herein incorporated by reference in its entirety) and ichthyosis (Paller A S et al., J Allergy Clin Immunol 139:152-165 (2017) which is herein incorporated by reference in its entirety).

In some embodiments, compounds that can be evaluated using the rodents disclosed include candidate inhibitors of the IL-36 signaling, for example, but not limited to, a small molecule inhibitor, a nucleic acid-based inhibitor (e.g., siRNA, ribozyme, antisense construct, etc.), an antigen-binding protein (e.g., antibody or antigen-binding fragment thereof), or a blocking peptide/peptide inhibitor.

In some embodiments, a candidate inhibitor is an antibody or antigen-binding fragment thereof. Both monoclonal and polyclonal antibodies are suitable to be tested in a rodent disclosed herein. In some embodiments, an antibody specifically binds to a human IL-36R protein. In some embodiments, an antibody specifically binds to the IL1RL2 subunit of a human IL-36R protein.

Candidate compounds can be evaluated by inducing inflammation, e.g., IMQ induced skin inflammation or DSS-induced intestinal inflammation, in a rodent disclosed herein, and determining whether a candidate compound can treat or inhibit the induced inflammation. The term “treating” or “inhibiting” includes ameliorating the severity, slowing down the progression, eliminating, delaying or preventing the onset of the induced inflammation and symptoms, or a combination thereof.

In some embodiments, a rodent is administered with a candidate compound prior to, together with, or after administration of an agent that induces inflammation. Candidate compounds may be dosed via any desired route of administration including parenteral and non-parenteral routes of administration. Parenteral routes include, e.g., intravenous, intraarterial, intraportal, intramuscular, subcutaneous, intraperitoneal, intraspinal, intrathecal, intracerebro ventricular, intracranial, intrapleural or other routes of injection. Non-parenteral routes include, e.g., oral, nasal, transdermal, pulmonary, rectal, buccal, vaginal, ocular. Administration may also be by continuous infusion, local administration, sustained release from implants (gels, membranes or the like), and/or intravenous injection.

In some embodiments, suitable control rodents can include, e.g., humanized rodents without being subjected to an induced inflammation; humanized rodents subjected to an induced inflammation without any compound or with a control compound not expected to have any therapeutic efficacy (e.g., an isotype control antibody); and humanized rodents subjected to an induced inflammation and a compound known to be therapeutically effective.

To assess the efficacy of a candidate compound on skin inflammation, the compound can be administered to the rodent before, during or after the IMQ treatment. In specific embodiments, a candidate compound is administered subcutaneously at or near the skin area where IMQ is applied. A compound is considered to be effective if it inhibits skin inflammation as compared to control rodents not administered the compound. For example, a compound is considered effective if the total pathology score is reduced by 10%, 15%, 20%, 30%, 40%, 50%, 60%, 70%, 80%, 90% or more (e.g., reduced by 20%-90%, 20%-80%, 20%-70%, 20%-60%, 20%-50%, 20%-40%, 30%-90%, 30%-80%, 30%-70%, 30%-60%, 30%-50%, or 30%-40%); or if the concentration(s) of one or more proinflammatory mediators are reduced by 10%, 15%, 20%, 30%, 40%, 50%, 60%, 70%, 80%, 90% or more (e.g., reduced by 20%-90%, 20%-80%, 20%-70%, 20%-60%, 20%-50%, 20%-40%, 30%-90%, 30%-80%, 30%-70%, 30%-60%, 30%-50%, or 30%-40%).

To assess the efficacy of a candidate compound on colon inflammation, the compound can be administered to the rodent before, during or after the DSS or oxazolone treatment. In some embodiments, a candidate compound is administered intraperitoneally a number of days (e.g., 5, 6, 7, 8, or 9 days) after the DSS treatment has started. In some embodiments, a candidate compound is administered intraperitoneally multiple times during the oxazolone treatment; for example, at 2-3 days after a topical application of oxazolone, at one or more of the intrarectal administrations of oxazolone (e.g., at first intrarectal administration of oxazolone, and at third intrarectal administration of oxazolone). A compound is considered to be effective if it inhibits colitis as compared to control DITRA rodents not administered the compound. For example, a compound is considered effective if the total pathology score is reduced by 10%, 15%, 20%, 30%, 40%, 50%, 60%, 70%, 80%, 90% or more (e.g., reduced by 20%-90%, 20%-80%, 20%-70%, 20%-60%, 20%-50%, 20%-40%, 30%-90%, 30%-80%, 30%-70%, 30%-60%, 30%-50%, or 30%-40%); if the concentration of one or more proinflammatory mediators are reduced by 10%, 15%, 20%, 30%, 40%, 50%, 60%, 70%, 80%, 90% or more (e.g., reduced by 20%-90%, 20%-80%, 20%-70%, 20%-60%, 20%-50%, 20%-40%, 30%-90%, 30%-80%, 30%-70%, 30%-60%, 30%-50%, or 30%-40%); if the fecal Lcn2 is reduced by 10%, 15%, 20%, 30%, 40%, 50%, 60%, 70%, 80%, 90% or more (e.g., reduced by 20%-90%, 20%-80%, 20%-70%, 20%-60%, 20%-50%, 20%-40%, 30%-90%, 30%-80%, 30%-70%, 30%-60%, 30%-50%, or 30%-40%); if the MPO activity in colon homogenates is reduced by 10%, 15%, 20%, 30%, 40%, 50%, 60%, 70%, 80%, 90% or more (e.g., reduced by 20%-90%, 20%-80%, 20%-70%, 20%-60%, 20%-50%, 20%-40%, 30%-90%, 30%-80%, 30%-70%, 30%-60%, 30%-50%, or 30%-40%); if the colon length is increased by 10%, 15%, 20%, 30%, 40%, 50%, 60%, 70%, 80%, 90% or more (e.g., reduced by 20%-90%, 20%-80%, 20%-70%, 20%-60%, 20%-50%, 20%-40%, 30%-90%, 30%-80%, 30%-70%, 30%-60%, 30%-50%, or 30%-40%); or a combination thereof.

The present description is further illustrated by the following examples, which should not be construed as limiting in any way. The contents of all cited references (including literature references, issued patents, and published patent applications as cited throughout this application) are hereby expressly incorporated by reference in their entireties.

### Example 1. Generation of A Quadruple-Humanized Mouse Strain

Two genetically-engineered mouse strains were created using VelociGene® technology (Poueymirou et al., Nat Biotechnol. 2007 January; 25(1): 91-9; Valenzuela et al., Nat Biotechnol 2003 June; 21:652-59, which are herein incorporated by reference in their entireties): an Il1rl2 single-humanized mouse strain, and a Il1f6, Il1f8, Il1f9 triple humanized mouse strain. A “quadruple-humanized” strain was generated by breeding the single-humanized and triple-humanized strains together.

### Generation of A Single-Humanized Il1rl2 Mouse Strain

A “single-humanized” strain was generated in which the portion of the mouse Il1rl2 gene encoding the extracellular domain of mouse Il1rl2 (interleukin-1 receptor-like 2 protein) was replaced with a fragment of the human IL1RL2 gene encoding the corresponding extracellular domain of human IL1RL2 (Il1rl2 IL1RL2 exons 3-8, with intervening introns and parts of the flanking introns, respectively) (FIG. 1A). The resulting humanized gene encodes a chimeric receptor that maintains the intracellular signaling specificity of mice, while rendering the extracellular domain human and able to bind human ligands IL1F6, IL1F8, and IL1F9, also called IL36A, B and G, respectively. Homozygous humanized Il1rl2 mice are referred to as Il1rl2^{hu/hu}.

More specifically, mouse bacterial artificial chromosome (BAC) clone RP23-235L22 containing a mouse Il1rl2 gene was used and modified as follows to provide a targeting vector. A DNA fragment was generated to include a 5′ mouse homology nucleotide sequence, a human IL1RL2 genomic DNA of about 32,389 bp (containing 3′ 346 bp of intron 2, exons 3-8 with all intervening introns, and 5′ 1101 bp of intron 8), a self-deleting neomycin cassette of about 4,996 bp, and a 3′ mouse homology sequence (FIG. 1B). This DNA fragment was used to modify BAC clone RP23-235L22 through homologous recombination in bacterial cells. As a result, an ectodomain-encoding mouse Il1rl2 genomic fragment (of about 25,324 bp, including the 3′ 155 bp of mouse intron 2, exons 3 through 8 with all intervening introns, and the 5′ 642 bp of mouse intron 8) in the BAC clone was replaced with the human IL1RL2 genomic DNA, followed by the self-deleting neomycin cassette (FIG. 1B). The resulting modified BAC clone included, from 5′ to 3′, (i) a 5′ mouse homology arm containing about 112.2 kb of mouse genomic DNA, mouse Il1rl2 exons 1-2 and a 5′ portion of intron 2; (ii) a human IL1RL2 genomic fragment including a 3′ portion of intron 2, exons 3 through 8, and a 5′ portion of intron 8; (iii) a self-deleting neomycin cassette of about 4,996 bp, followed by (iv) a 3′ mouse homology arm of 31.3 kb containing the remaining mouse Il1rl2 exons downstream of exon 8, all the intervening introns and the 3′ UTR. See FIG. 1B. The junction sequences are also set forth at the bottom of FIG. 1B.

The modified BAC clone containing the humanized Il1rl2 gene, as described above, was used as a targeting vector to electroporate mouse F1H4 embryonic stem cells (50% C57BL/6NTac/50% 129S6/SvEvTac) to create modified ES cells comprising a humanized Il1rl2 gene. Positively targeted ES cells containing a humanized Il1rl2 gene were identified by an assay (Valenzuela et al., supra) that detected the presence of the human IL1RL2 sequences and confirmed the loss and/or retention of mouse Il1rl2 sequences. The primers and probes that were used to confirm humanization as described above were set forth in Table 5. Once a correctly targeted ES cell clone has been selected, the neomycin selection cassette can be excised. The humanized Il1rl2 locus after the deletion of the cassette is depicted in FIG. 1C, with the junction sequences also shown in FIG. 1C.

TABLE 5
Mouse TaqMan Loss of allele assays

| | | |
|---|---|---|
| 7252mTU | Fwd | GGGTCCATTATTTGAGACTTTCCA |
| | | (SEQ ID NO: 34) |
| | Probe | AGCTTTCTGCTGGCCCCATTACTTG |
| | (BHQ) | (SEQ ID NO: 35) |
| | Rev | GCATGTGCCTGTCTTCACA |
| | | (SEQ ID NO: 36) |
| 7252mTD | Fwd | GAGTGGTACAACCTCTACATTTGAG |
| | | (SEQ ID NO: 37) |
| | Probe | CTGCAGCCTTCCCTGGTTGGTTC |
| | (BHQ) | (SEQ ID NO: 38) |
| | Rev | AGTGGGCAGCTCCCTTTAGA |
| | | (SEQ ID NO: 39) |

Human TaqMan Gain of allele assays

| | | |
|---|---|---|
| 7252hTU | Fwd | CCCACTGCATGTGAAGCAT |
| | | (SEQ ID NO: 40) |
| | Probe | TTGAGTGTCTGCACCTGAGCCA |
| | (BHQ) | (SEQ ID NO: 41) |
| | Rev | GGAGACCTCTTATTAGCCTGTGA |
| | | (SEQ ID NO: 42) |
| 7252hTD | Fwd | CACCTGCCCTAAGTCATCTC |
| | | (SEQ ID NO: 43) |
| | Probe | TACAGCAAGAGCAGAGGCCACA |
| | (BHQ) | (SEQ ID NO: 44) |
| | Rev | GCCTGCAGAGAAGCAATGTTC |
| | | (SEQ ID NO: 45) |

Selected ES cell clones were microinjected into 8-cell embryos from Charles River Laboratories Swiss Webster albino mice, yielding F0 VelociMice® that were 100% derived from the targeted cells (Poueymirou et al. 2007, supra). Mice bearing a humanized locus were again confirmed and identified by genotyping of DNA isolated from tail snips using a modification of allele assay (Valenzuela et al., supra) that detected the presence of the human gene sequences. Animals homozygous for a humanized locus were made by crossing heterozygous animals.

An alignment of the amino acid sequences of the resulting humanized/chimeric Il1rl2 receptor (SEQ ID NO: 7), mouse Il1rl2 protein (SEQ ID NO: 4), and human IL1RL2 protein (SEQ ID NO: 2), is provided in FIG. 1D.

### Generation of A Triple-Humanized Mouse Strain

A “triple-humanized” strain was generated in which the complete coding sequence of each of the mouse Il1f6, Il1f8, and Il1f9 genes was replaced with the complete coding sequence of each of the human IL1F6, IL1F8, and IL1F9 genes, respectively. This strategy resulted in humanized genes encoding human ligands that are able to bind the human extracellular domain of the chimeric Il1rl2 receptor. Homozygous humanized Il1f6, Il1f8, Il1f9 mice are referred to as Il1f6^{hu/hu}, 111f8^{hu/hu}, 111f9^{hu/hu}.

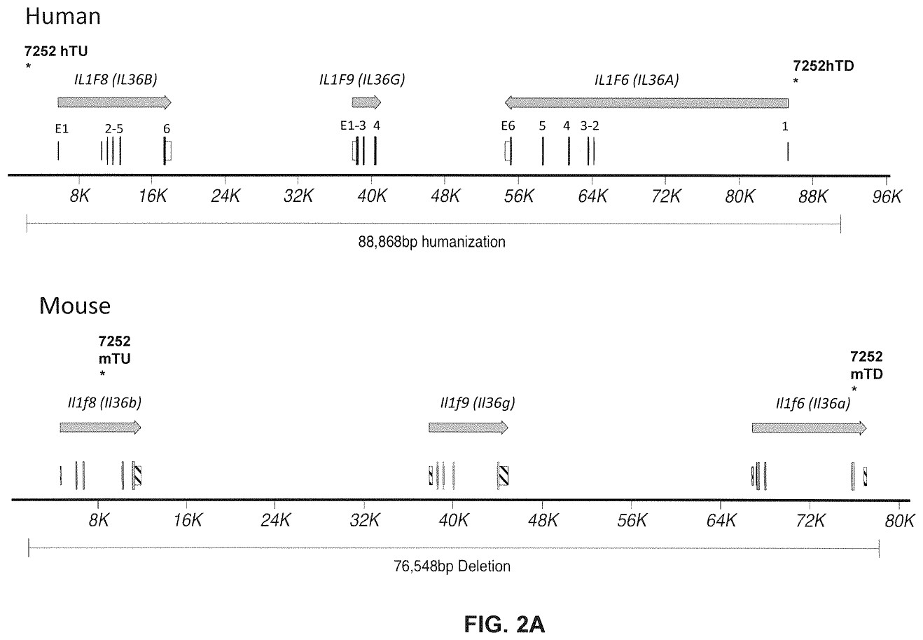

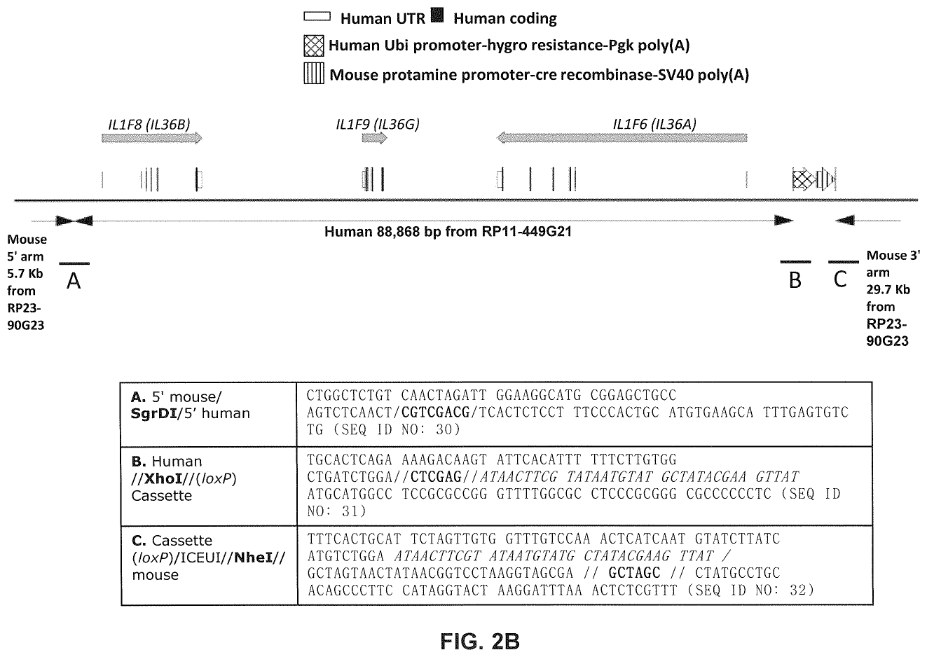

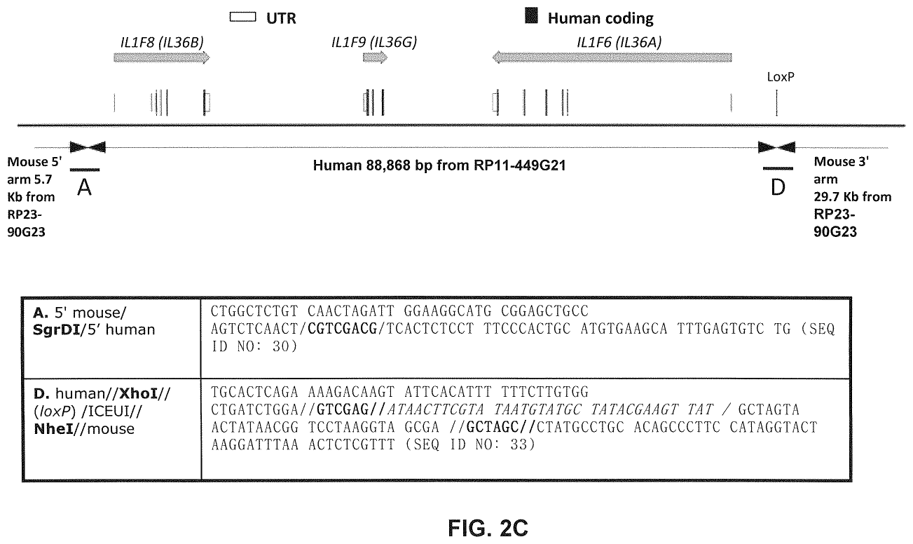

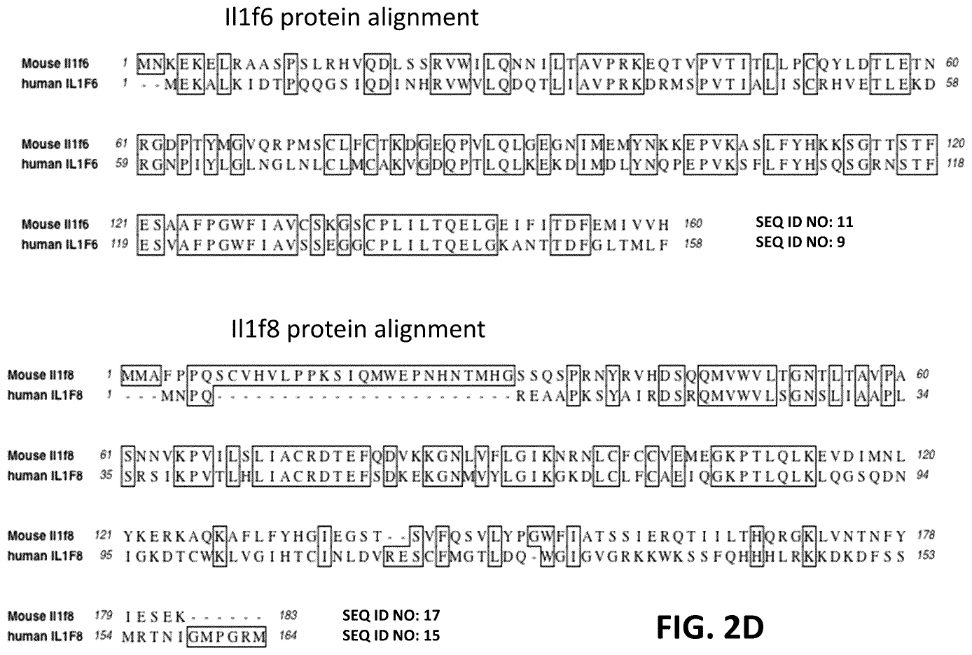

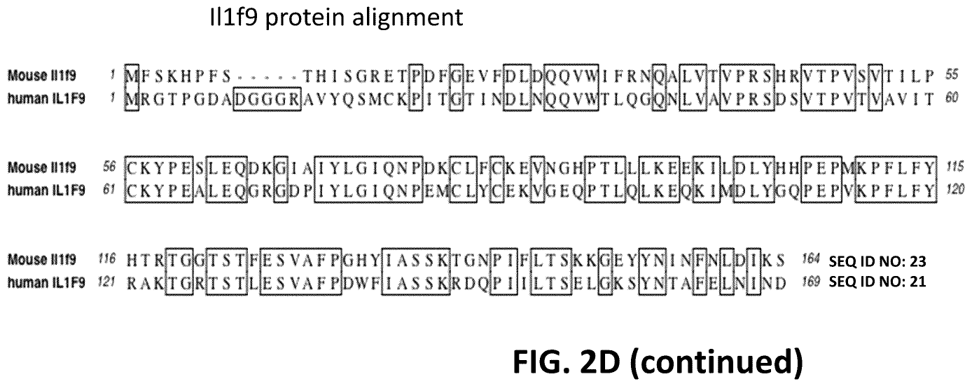

More specifically, mouse bacterial artificial chromosome (BAC) clone RP23-90G23 containing mouse Il1f8, Il1f9, and Il1f6 genes and intergenic sequences was used and modified as follows to provide a targeting vector. A DNA fragment was generated to include a 5′ mouse homology nucleotide sequence, a human genomic DNA of about 88,868 bp (containing the promoters, untranslated regions, and coding sequences for human IL1F8, IL1F9, and IL1F6), a self-deleting hygromycin cassette of about 5,218 bp, and a 3′ mouse homology sequence (FIG. 2B). This DNA fragment was used to modify BAC clone RP23-90G23 through homologous recombination in bacterial cells. As a result, a mouse genomic fragment of about 76,548 bp in the BAC clone was replaced with the human genomic DNA, followed by the self-deleting cassette (FIGS. 2A-2B). The resulting modified BAC clone included, from 5′ to 3′, (i) a 5′ mouse homology arm containing about 5.7 kb of mouse genomic DNA, (ii) a human genomic fragment of about 88,868 bp (containing the promoters, untranslated regions, and coding sequences for human IL1F8, IL1F9, and IL1F6), (iii) a self-deleting hygromycin cassette of about 5,218 bp, and (iv) a 3′ mouse homology sequence of about 29.7 kb. See FIG. 2B. The junction sequences are also set forth at the bottom of FIG. 2B.

The modified BAC clone containing the human IL1F8, IL1F9, and IL1F6 gene sequences, as described above, was used as a targeting vector to electroporate mouse F1H4 embryonic stem cells (50% C57BL/6NTac/50% 129S6/SvEvTac) to create modified ES cells comprising human IL1F8, IL1F9, and IL1F6 gene sequences. Positively targeted ES cells containing human IL1F8, IL1F9, and IL1F6 gene sequences were identified by an assay (Valenzuela et al., supra) that detected the presence of the human sequences and confirmed the loss and/or retention of mouse sequences. The primers and probes that were used to confirm humanization as described above were set forth in Table 6. Once a correctly targeted ES cell clone has been selected, the hydromycin selection cassette can be excised. The humanized locus after the deletion of the cassette is depicted in FIG. 2C, with the junction sequences also shown in FIG. 2C.

TABLE 6
Mouse TaqMan Loss of allele assays

| | | |
|---|---|---|
| 7210U | Fwd | CAGCCGCTACACACCACAA |
| | | (SEQ ID NO: 46) |
| | Probe | CCAGCTGCTACACAAATGCAGGGC |
| | (BHQ) | (SEQ ID NO: 47) |
| | Rev | CGTCATCTCCTGCCAGTTCA |
| | | (SEQ ID NO: 48) |
| 7210D | Fwd | CTGCTGCTCAGAGCATTGAAA |
| | | (SEQ ID NO: 49) |
| | Probe | CCATGGCCAGGGAAGGCTTACTA |
| | (BHQ) | (SEQ ID NO: 50) |
| | Rev | CGCCTCCGACTGAACATATGAC |
| | | (SEQ ID NO: 51) |

Human TaqMan Gain of allele assays

| | | |
|---|---|---|
| 7210hU | Fwd | CCTGATATGCATCTTTCCCTATGGA |
| | | (SEQ ID NO: 52) |
| | Probe | ATGGCACCTCAGACCAGACCCAC |
| | (BHQ) | (SEQ ID NO: 53) |
| | Rev | GGCAGCAATAATACTGGGACAAAC |
| | | (SEQ ID NO: 54) |
| 7210hD | Fwd | GGCCAGGAAACACATCTGAAG |
| | | (SEQ ID NO: 55) |
| | Probe | AGCAGTGACTCTAAATGCTCAGTGTGA |
| | (BHQ) | (SEQ ID NO: 56) |
| | Rev | GCCTCACTCCAACAACATTATGA |
| | | (SEQ ID NO: 57) |

Selected ES cell clones were microinjected into 8-cell embryos from Charles River Laboratories Swiss Webster albino mice, yielding F0 VelociMice® that were 100% derived from the targeted cells (Poueymirou et al. 2007, supra). Mice bearing a humanized locus were again confirmed and identified by genotyping of DNA isolated from tail snips using a modification of allele assay (Valenzuela et al., supra) that detected the presence of the human gene sequences. Animals homozygous for a humanized locus were made by crossing heterozygous animals.

### Generation of A Triple-Humanized Mouse Strain

A “quadruple-humanized” strain was generated by crossing the single-humanized and triple-humanized strains together, bred to homozygosity for both loci on a 100% C57BL/6NTac background. Homozygous quadruple-humanized strain is referred to as Il1rl2^{hu/hu} Il1f6^{hu/hu} Il1f8^{hu/hu} Il1f9^{hu/hu}, or a “DITRA-like” mouse. Neo and hygro cassettes were deleted in the F0 germline by self-deleting technology.

### Example 2. Characterization of DITRA Mice

### Materials and Methods

Acute and chronic IMQ-induced skin inflammation induction and antibody treatment in DITRA-like mice—To induce skin inflammation, 8-10 weeks old humanized DITRA-like female mice had their back hair shaved using mouse hair trimmer (Oster, MiniMax, Cat #78049-100) and skin depilated with 0.5 g Veet hair removal gel three days prior to IMQ cream application. A daily topical dose of 62.5 mg of commercially available IMQ cream (5%) (Aldara, GM Health Care Limited, NDC 99207-206-12, lot #QJ044A) or Vaseline (CVS Pharmacy, NDC 59779-902-88) was applied on the shaved back skin of the mice for four consecutive days for acute and nine days for chronic disease induction. A daily topical dose of 62.5 mg of Aldara translated into a daily dose of 3.125 mg of an active compound. In acute IMQ-induced skin inflammation, an anti-human IL-36R antibody was administered subcutaneously into back skin at 10 mg/kg 3 days before and 1 day after starting IMQ application. Control group received PBS and 10 mg/kg of hIgG4 Isotype control injections. In chronic IMQ-induced skin inflammation, the same anti-human IL-36R antibody was administered subcutaneously into back skin at 10 mg/kg therapeutically on days 4 and 8 after starting IMQ application. Two or three days after treatments, the back skin of mice started to display signs of erythema, scaling and thickening. The severity of inflammation was measured on a daily basis using an adapted version of the clinical Psoriasis Area and Severity Index. Erythema, scaling and thickening were scored independently on a scale from 0-4:0, none; 1, slight; 2, moderate; 3, marked; and 4, very marked (van der Fits et al., J Immunol 2009, 182:5836-5845, which is herein incorporated by reference in its entirety). On day 4 of acute and day 11 of chronic IMQ-induced skin inflammation, skin thickness was measured using caliper (Kaefer).

Histopathology—Skin tissues of 6 mm diameter from murine back were fixed in 10% buffered formalin, and 4-5 μm paraffin embedded sections were stained with hematoxylin and eosin. Skin sections were evaluated blindly for the presence of parakeratosis, orthokeratosis, Munro's microabscess, acanthosis, epidermal ulceration, inflammation in the dermis and hypodermis, blood vessel congestion in the dermis and hypodermis, follicular hyperkeratosis and epithelial hyperplasia. A 0-4 scoring scale was used: 0—within normal limits, 1—minimal, 2—mild, 3—moderate and 4—severe (van der Fits et al., J Immunol 2009, 182:5836-5845, which is incorporated herein by reference in its entirety). A total pathology score was calculated for each mouse by adding the individual histopathological feature scores. Data analysis was performed using GraphPad Prism™ software.

Measurement of cytokines in skin homogenates—Full thickness skin tissues of 6 mm diameter from murine back were taken and placed in 15 mL tube containing T-per buffer (Thermo Scientific, Cat #378510, lot #RF236217), 1× Halt Protease Inhibitor Cocktail (Thermo Scientific, Cat #87786, lot #QG221763) and 5M EDTA Solution (Thermo Scientific, Cat3 78429). Skin tissues were disrupted at 28000 rpm for 1 minute using a Polytron (PT10-35 GT-D, Cat #9158158) and put on ice. Generated skin homogenates were centrifuged at 1500 rpm for 8 minutes at 4° C. and the supernatants were collected into 96-well plates. Skin homogenates were subjected to Bradford protein assay using protein assay dye (BioRad, Cat #500-0006, lot #210008149) to quantify the total protein content. Cytokine concentrations in the skin homogenates were measured using a Proinflammatory Panel 1 (mouse) multiplex immunoassay kit (MesoScale Discovery, Cat #K15048D) according to manufacturer's instructions. In brief, 50 μL/well of calibrators and samples (diluted in Diluent 41) were added to the plates pre-coated with capture antibodies and incubated at room temperature while shaking at 700 rpm for 2 hours. The plates were then washed 3 times with 1×PBS containing 0.05% (w/v) Tween-20, followed by the addition of 25 μL of Detection Antibody Solution diluted in Diluent 45. After 2-hour incubation at room temperature while shaking, the plates were washed 3 times, and 150 μL of 2× Read Buffer was added to each well. Electrochemiluminescence was immediately read on a MSD Spector® instrument. Data analysis was performed using GraphPad Prism™ software. Cytokine levels were normalized to total protein content.

Induction of DSS-induced model of chronic colitis and antibody treatment in DITRA-like mice—To induce chronic DSS-mediated colitis, female DITRA-like mice aged 12-20 weeks with an average body weight of more than 23 g were given 1.5-3% DSS (Sigma-Aldrich Cat #87786, lot #PJ203966B) in drinking water for 7 days followed by distilled water for 11-13 days. A second cycle of DSS treatment (for 4 days) followed by water (5-6 days) was performed until day 27-30. Control group received distilled water for the duration of the study. Anti-human IL-36R and mIL-12p40 (Bioxcell Cat #BE0051, clone C17.8) antibodies were administered intraperitoneally at 10 mg/kg be-weekly starting on day 7. Control group received PBS and respective hIgG4 and ratIgG2a (Bioxcell Cat #BE0089, clone 2A3) Isotype control injections at 10 mg/kg. Mice were weighted and monitored for clinical signs of colitis (e.g. stool consistency and fecal blood) on a daily basis. On day 27-30, mice were euthanized and colon lengths were measured. To evaluate DSS colitis, the following features were scored: inflammation (severity and extent), epithelial changes (erosion/ulcer), changes in the crypts (crypt loss, cryptitis/crypt abscess, regeneration/hyperplasia, goblet cell loss), submucosal edema and percentage of tissue area with pathology relative to the total tissue area on the slide. A 0-4 scoring scale was used: 0-O-within normal limits, 1-minimal, 2-mild, 3-moderate and 4-severe. A total pathology score was calculated for each mouse by adding the individual histopathological feature scores.

Measurement of Lcn-2 in fecal samples—To monitor intestinal inflammation throughout the study, feces from individual DITRA-like mice were collected into 2 mL deep well plates on a weekly basis and stored at −80° C. Upon the completion of the study, feces collected on different days were subjected to homogenization. In brief, fecal samples were reconstituted with 1 mL PBS containing 0.1% Tween-20, 1× Halt Protease Inhibitor Cocktail (Thermo Scientific, Cat #87786, lot #QG221763) and 5M EDTA Solution (Thermo Scientific, Cat3 78429). After adding 2 Tungsten 3 mm Carbide Beads to the wells (Qiagen, Cat #69997), the plates were placed on a shaker at highest speed overnight at 4° C. Homogenous fecal suspensions were centrifuged at 1200 rpm for 10 minutes at 4° C. and the supernatants were collected into 96-well plates. Fecal Lipocalin-2 (Lcn2) levels were measured using mouse Duoset Lipocalin-2/NGAL ELISA kit (R&D Systems, Cat #DY1857, lot #P116359) according to manufacturer's instructions. Data analysis was performed using GraphPad Prism™ software.

Measurement of Myeloperoxidase (MPO) activity in colon homogenates—Pieces of distal part of the colon were taken into 2 mL microcentrifuge tubes containing 2 Tungsten 3 mm Carbide Beads (Qiagen, Cat #69997) containing T-per buffer (Thermo Scientific, Cat #378510, lot #RF236217), 1× Halt Protease Inhibitor Cocktail (Thermo Scientific, Cat #87786, lot #QG221763) and 5M EDTA Solution (Thermo Scientific, Cat #78429). Colon tissues were disrupted using Qiagen Tissue Lyser II at frequency of 27.5 s⁻¹ for 10 minutes. Tubes were centrifuged at 1500 rpm for 8 minutes at 4° C. and the supernatants were collected into 96-well plates. Colon homogenates were subjected to Bradford protein assay using protein assay dye (BioRad, Cat #500-0006, lot #210008149) to quantify the total protein content. Myeloperoxidase (MPO) activity in the colon homogenates was measured using mouse MPO ELISA Kit (Hycult Biotech, Cat #HK210-02, lot #21022KO617-Y) according to manufacturer's instructions. Data analysis was performed using GraphPad Prism™ software. MPO levels were normalized to total protein content.

Measurement of cytokines in colon homogenates—Cytokine concentrations in the colon homogenates were measured using a Proinflammatory Panel 1 (mouse) multiplex immunoassay kit (MesoScale Discovery, Cat #K15048D) according to manufacturer's instructions. In brief, 50 μL/well of calibrators and samples (diluted in Diluent 41) were added to the plates pre-coated with capture antibodies and incubated at room temperature while shaking at 700 rpm for 2 hours. The plates were then washed 3 times with 1×PBS containing 0.05% (w/v) Tween-20, followed by the addition of 25 μL of Detection Antibody Solution diluted in Diluent 45. After 2-hour incubation at room temperature while shaking, the plates were washed 3 times, and 150 μL of 2× Read Buffer was added to each well. Electrochemiluminescence was immediately read on a MSD Spector® instrument. Data analysis was performed using GraphPad Prism™ software. Cytokine levels were normalized to total protein content.

Statistical analysis-Statistical significance within the groups was determined by one-way Anova with Tukey's multiple comparison post-test (*p<0.05, **p<0.005, ***p<0.0005, ****p<0.0001) (same for both *—statistical significance from PBS-treated group and #—statistical significance from corresponding Isotype-treated group).

### Results

### Humanized DITRA-Like Mice Exhibit Enhanced Skin Inflammation in IMQ-Induced Model of Psoriasis

To interrogate the role of deregulated IL-36R signaling similar to GPP or DITRA (for “Deficiency of Interleukin Thirty-six Receptor Antagonist”) patients, mice were generated to have humanized IL-36R (e.g., Il1rl2) and human IL-36α, β, γ ligands, but not the IL-36Ra antagonist, as described in Example 1. The resulting hIL-36R/hIL-36α, β, γ mice were referred to as DITRA-like mice due to a 20-fold decreased affinity of mouse IL-36Ra for human IL-36R leading to an enhanced IL-36R signaling similar to DITRA patients (for a more detailed discussion of DITRA patients, see, e.g., Marrakchi et al., N Engl J Med 2011, 365:620-628, which is herein incorporated by reference in its entirety).

In an unchallenged state, DITRA-like mice did not develop any spontaneous diseases. In contrast, in the preclinical model of IMQ-induced psoriasiform dermatitis that closely resembles human psoriasis lesions in terms of the phenotypic and histological characteristics (for a more detailed discussion regarding characterization of psoriasiform dermatitis, see van der Fits et al., J Immunol 2009, 182:5836-5845; Swindell et al., PLOS One 2011, 6: e18266, all of which are herein incorporated by reference in their entireties), DITRA-like mice displayed an enhanced skin inflammation compared to their WT littermates (FIGS. 3A-3B). In brief, IMQ was applied daily to the shaved back skin of DITRA-like and WT mice for four consecutive days. On day 5 skin was harvested for subsequent histopathological evaluation, protein and RNA isolation. IMQ-treated DITRA-like mice developed more severe psoriasis lesions such as scaling, erythema and skin thickening compared to WT littermates (FIG. 3A). Consistent with clinical features, histopathological evaluation of the skin revealed more enhanced acanthosis, parakeratosis and disturbed keratinocyte differentiation in DITRA-like mice (FIG. 3B). IMQ-treated DITRA-like mice also displayed significantly increased numbers of Munro's abscesses or pustules similarly to patients with GPP. Moreover, IMQ application led to increased levels of pro-inflammatory molecules deregulated in psoriasis (pro-inflammatory molecules including, e.g., IL-17a, IL-17f, IL-23a, S100A8, Defb4) in the skin of DITRA-like mice compared to WT littermates (FIG. 3C). Further, IL-36α, IL-36β, and IL-36γ mRNA expression were equally increased in IMQ-treated DITRA-like mice and IMQ-treated wild type mice as compared to Vaseline treated DITRA-like mice and Vaseline treated wild type mice, respectively; however, protein levels of IL-36α and IL-36β cytokines were detected at 2-fold higher concentrations in the inflamed skin of DITRA-like mice as compared to the levels in the inflamed skin of wild type mice.

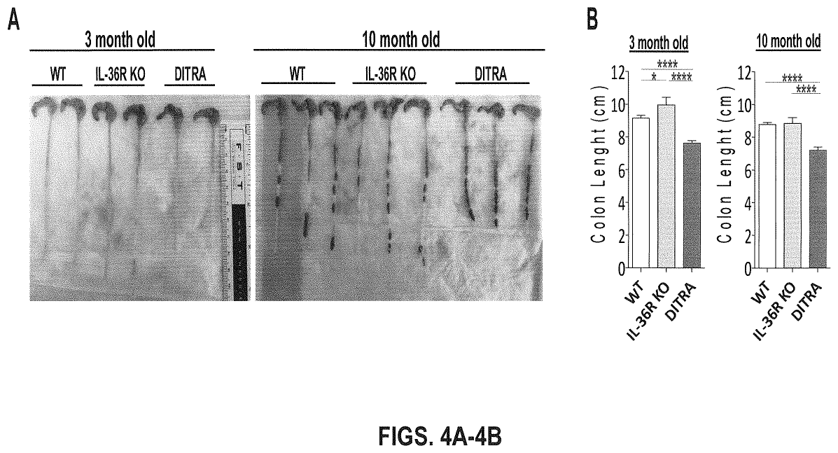

Humanized DITRA-like mice display a defect in mucosal healing in DSS-induced model of chronic colitis—In recent years, several studies in subsets of patients demonstrated deregulated expression of IL-36 axis in IBD and its possible contribution to the intestinal inflammation. Expression of IL-36α and IL-36γ was shown to be elevated in inflamed mucosa of patients with ulcerative colitis (see Medina-Contreras et al., J Immunol 2016, 196:34-38; Nishida et al., Inflamm Bowel Dis 2016, 22:303-314, and Russell, Mucosal Immunol 2016, 9:1193-1204, which are all herein incorporated by reference in their entireties). In preclinical models, IL-36R deficiency protected from DSS- and Oxazolone-induced colitis (Medina-Contreras et al., J Immunol 2016, 196:34-38; Harusato et al., Mucosal Immunol 2017, 10:1455-1467, which are all herein incorporated by reference in their entireties). At steady state, DITRA-like mice did not develop spontaneous ileitis/colitis, but displayed significantly shortened colons at both young and old age (3 and 10 month old, respectively) (FIGS. 4A and 4B). In addition, at steady state and as compared to wild type mice, DITRA-like mice exhibited elevated levels of IL-36 cytokines, as well as a trend towards increased IL-17F and IL-17A levels, and reduced IL-21 levels, in the colon.

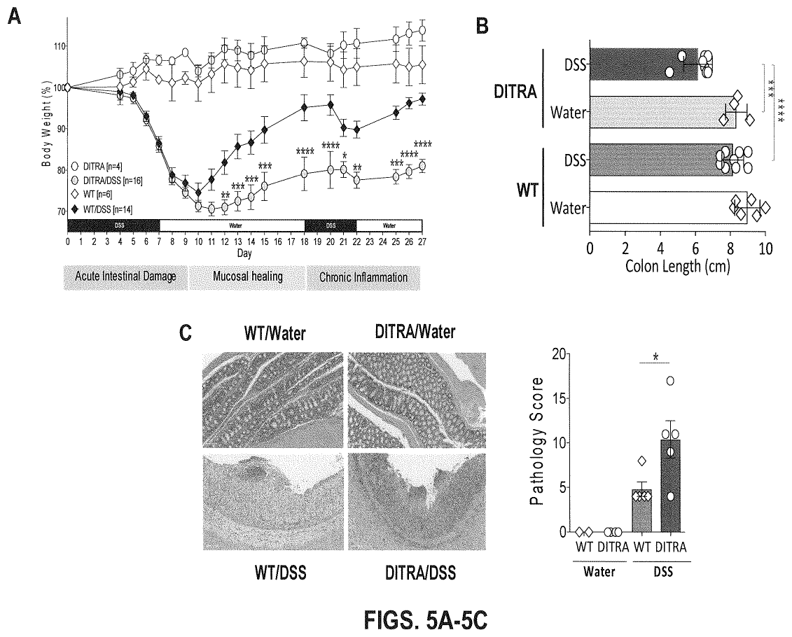

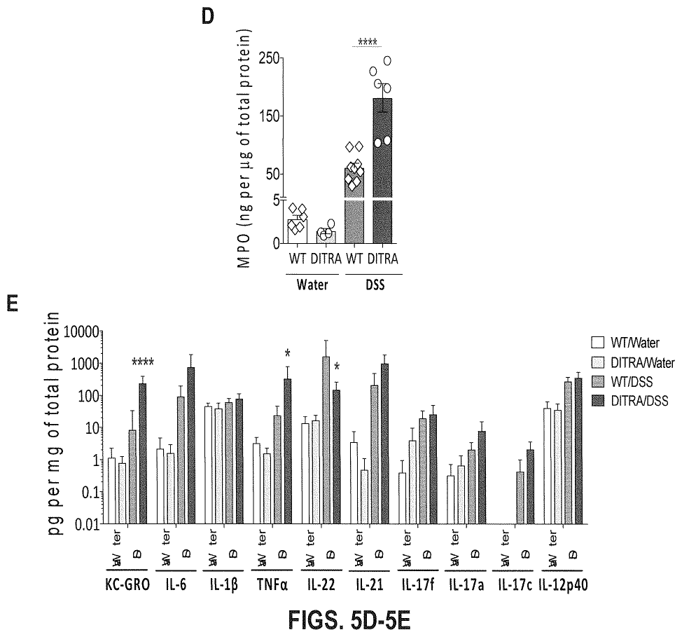

To evaluate the role of IL-36 axis in intestinal inflammation, a chemically induced model of intestinal injury was used by utilizing oral administration of DSS that damages the colonic epithelium (Okayasu et al., Gastroenterology 1990, 98:694-702, herein incorporated by reference in its entirety) and triggers potent inflammatory responses (Rakoff-Nahoum et al., Cell 2004, 118:229-241, herein incorporated by reference in its entirety) exhibiting main features of IBD, in particular ulcerative colitis. DITRA-like mice and their co-housed WT littermates were subjected to DSS-induced chronic colitis regimen by administration of 2.5% DSS for 7 days followed by 11 days of water (first cycle) and 1.5% DSS for 4 days followed by 5 days of water (second cycle). In the acute phase of the disease, DITRA-like mice developed intestinal inflammation similarly to co-housed WT littermates (FIG. 5A). Interestingly, in the repair phase of the disease, DITRA-like mice showed inability to recover from DSS-induced mucosal damage reflected in the sustained body weight loss (FIG. 5A), significantly reduced colon length (FIG. 5B), more severe pathology score (associated with more severe ulceration, extensive epithelial erosion and neutrophilic infiltration) (FIG. 5C), significantly increased fecal Lipocalin-2 (Lcn2) levels and myeloperoxidase activity (FIG. 5D), up-regulation of pro-inflammatory cytokines such as KC-GRO, TNF-α and IL-6, and a reduction in the IL-22 levels in the colon (FIG. 5E). In addition, DITRA-like mice exhibited a death rate of about 62.5% (data not shown). By contrast, WT mice did recover in the repair phase of colitis and had a death rate of 25%. Gut epithelial integrity was also investigated by orally gavaging DITRA-like mice and wild type mice with fluorescein isothiocyanate (FITC)-dextran. A significant increase in the FITC-dextran levels was observed in the sera of DITRA-like mice at day 14 of DSS treatment, indicating that the IL-36 pathway is involved in regulating intestinal permeability during gut injury. Thus, enhanced IL-36 signaling in DITRA-like mice leads to exacerbation of intestinal inflammation and a defect in mucosal repair suggesting the role of IL-36 in regulating the mechanisms involved in intestinal tissue remodeling.

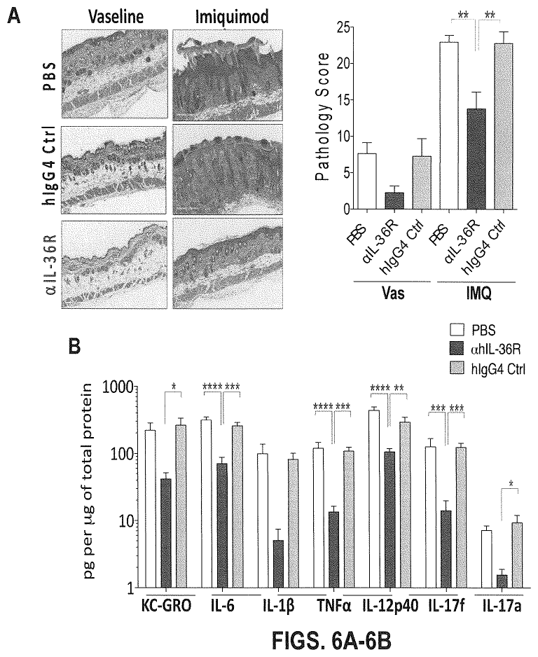

Anti-human IL-36R monoclonal antibody inhibits acute skin inflammation in DITRA-like mice at prophylactic dosing—To examine the role of IL-36R in skin inflammation, an anti-human IL-36R monoclonal antibody was tested in IMQ-induced model of psoriasiform dermatitis. IMQ was applied daily to the shaved back skin of DITRA-like mice for four consecutive days. The anti-human IL-36R monoclonal antibody was administered at 10 mg/kg 3 days before (−3d) and one day after (d1) starting the IMQ application. Control groups received PBS and hIgG4 Isotype control injections at 10 mg/kg. On day 5 skin was harvested for subsequent histopathological evaluation and protein isolation. The anti-human IL-36R monoclonal antibody significantly reduced IMQ-induced total pathology score including parakeratosis and Munro's microabscess compared to PBS- and Isotype control-treated groups (FIG. 6A). Human IL-36R blockade also resulted in 66-93% reduction in KC-GRO, IL-6 and TNFα (FIG. 6B). Importantly, the anti-human IL-36R antibody treatment significantly decreased levels of pro-inflammatory cytokines deregulated in psoriasis (e.g., IL-12p40, IL-17f and IL-17a) (FIG. 6B) suggesting a tightly regulated interplay between these cytokines pathways. The observed efficacy of the anti-human IL-36R monoclonal antibody in abrogating acute IMQ-induced skin inflammation was dose-dependent, with a dose of 10 mg/kg resulting in stronger inhibition of skin inflammation as compared to a dose of 1 mg/kg, as determined based on the pathology score, skin thickness, and levels of pro-inflammatory cytokines in skin homogenates (data not shown).

Anti-human IL-36R monoclonal antibody inhibits chronic skin inflammation at therapeutic dosing—To further examine the therapeutic efficacy of human IL-36R antagonism in vivo, the same anti-human IL-36R monoclonal antibody was tested in chronic IMQ-induced model of skin inflammation. For the duration of two weeks, IMQ was applied to the shaved back skin of DITRA-like mice for nine days in two rounds separated by two days without the treatments. The anti-human IL-36R monoclonal antibody was administered subcutaneously at days 5 and 9 after starting IMQ application at 10 mg/kg dose. Control groups received PBS and hIgG4 Isotype control injections at 10 mg/kg. On day 12 skin thickness was measured and tissues harvested for subsequent histopathological evaluation and protein isolation. Similar to acute IMQ-induced inflammation, prolonged IMQ application led to upregulation of proinflammatory mediators in the skin of DITRA-like mice (data not shown). The anti-human IL-36R monoclonal antibody showed a significant efficacy in reducing IMQ-induced skin thickness and pathology lesion scores in DITRA-like mice (FIGS. 7A and 7B, respectively). In addition, administration of the anti-human IL-36R monoclonal antibody led to a significant inhibition of IMQ-induced production of pro-inflammatory cytokines in the skin of DITRA-like mice (FIG. 7C).

Altogether, the data demonstrated prophylactic and therapeutic efficacy of an anti-human IL-36R antibody in ameliorating acute and chronic IMQ-induced skin inflammation in vivo.

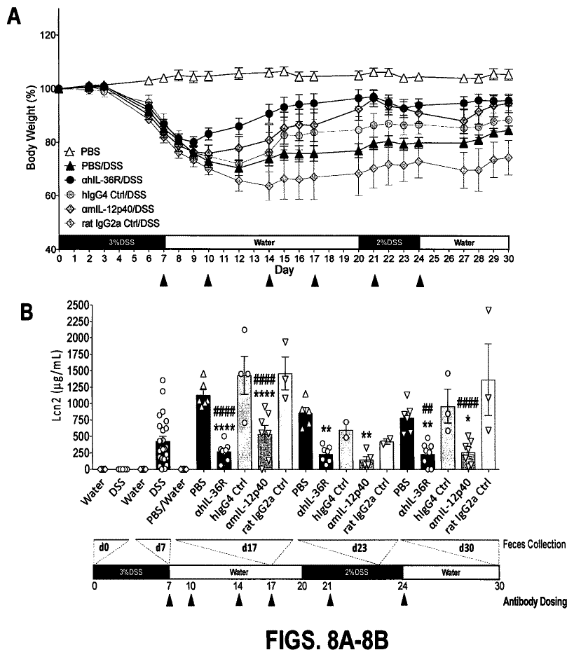

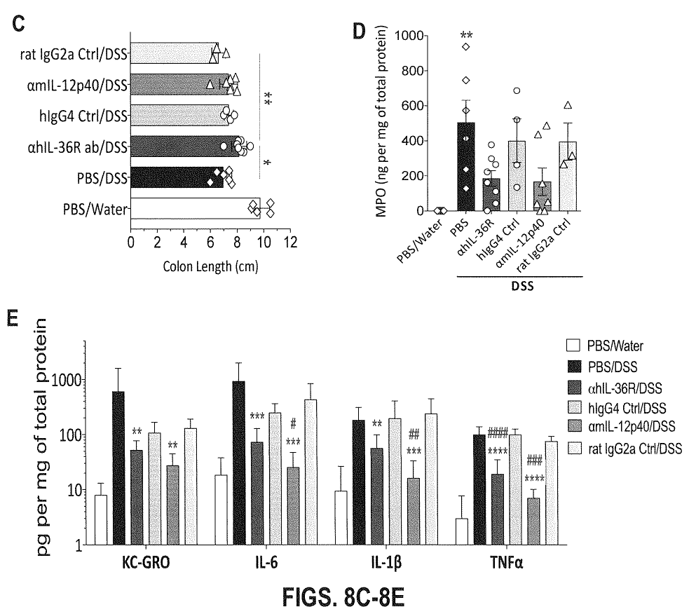

Anti-human IL-36R monoclonal antibody ameliorates DSS-induced chronic colitis in DITRA-like mice at therapeutic dosing—Having demonstrated the defect in mucosal healing as a result of enhanced IL-36 signaling in DITRA-like mice, it was further investigated as to whether IL-36 blockade would rescue the observed phenotype. DITRA-like mice were subjected to chronic DSS-induced colitis by administration of 3% DSS for 7 days followed by 13 days of water for two cycles. The same anti-human IL-36R monoclonal antibody and an anti-mIL-12p40 monoclonal antibody (a mouse surrogate for Ustekinumab which is approved for treating Crohn's disease) were administered at 10 mg/kg bi-weekly starting on day 7. Control groups received PBS and corresponding hIgG4 and rat IgG2a Isotype control injections at 10 mg/kg. Treatment with the anti-human IL-36R monoclonal antibody rescued the inability of DITRA-like mice to recover from DSS-induced mucosal damage and reduced the disease severity compared to PBS and Isotype control treatments in DITRA-like mice (FIG. 8A). To monitor the intestinal inflammation at different stages of the disease, feces from individual mice were collected on a weekly basis to measure fecal Lipocalin-2 (Lcn2) protein, a non-invasive biomarker of inflammation in intestinal injury (procedure described in, e.g., Thorsvik et al., J Gastroenterol Hepatol 2017, 32:128-135, which is herein incorporated by reference in its entirety). As shown in FIG. 8B, PBS-, hIgG4- and rat IgG2a-treated groups displayed significant upregulation of fecal Lcn2 levels on day 17, 23 and 30 compared to water alone. Administrations of the anti-human IL-36R monoclonal antibody surprisingly resulted in a significant reduction in Lcn2 levels compared to PBS- and Isotype-treated groups. Sustained reduction of fecal Lcn2 levels was observed in anti-human IL-36 antibody-treated groups at day 17, 23 and 30 (FIG. 8B).

Further results included that the hIL-36R blockade with the anti-human IL-36R monoclonal antibody led to an increase in colon length (FIG. 8C) and decrease in myeloperoxidase (MPO) activity (FIG. 8D) and 61-95% reduction in pro-inflammatory cytokines (FIG. 8E) in the colon of DSS-treated DITRA-like mice. The anti-human IL-36R monoclonal displayed comparable efficacy with IL-12p40 blockade in ameliorating DSS-induced chronic colitis as reflected in similarly decreased levels of fecal Lcn2, MPO activity and pro-inflammatory cytokines in the colon of DITRA-like mice (FIGS. 8A-8E).

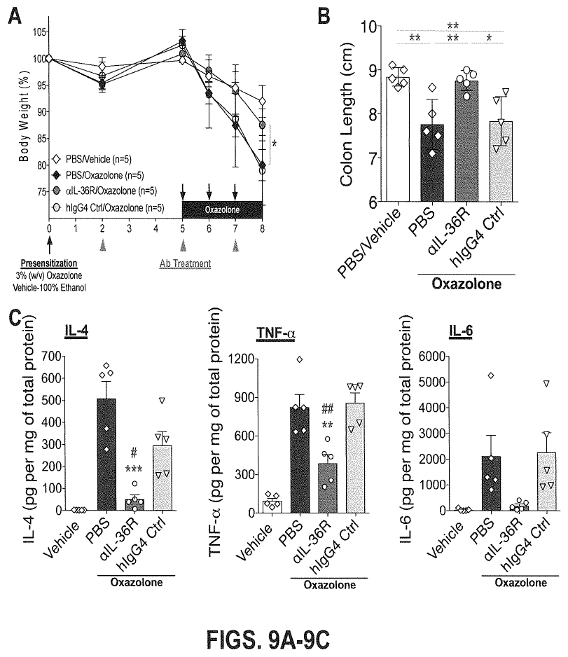

IL-36R blockade in oxazolone-induced colitis—The efficacy of IL-36R blockade was tested in oxazolone-induced colitis, another preclinical model of IBD with the histologic resemblance to human ulcerative colitis (Heller et al., Immunity 17, 629-638 (2002), which is incorporated in its entirety by reference). Prophylactic administration of anti-human IL-36R antibody significantly reduced oxazolone-induced disease severity in DITRA-like mice compared to PBS and isotype control treated groups, as reflected in average body weight loss and average colon length (FIGS. 9A-9B). In addition, IL-36R antagonism led to significantly reduced levels of IL-4 and TNF-α in the colon of oxazolone-treated DITRA-like mice (FIG. 9C).

Various publications, including patents, patent applications, published patent applications, accession numbers, technical articles and scholarly articles are cited throughout the specification. Each of these cited publications is incorporated by reference, in its entirety and for all purposes, in this document.

1.-45. (canceled)

46. A combination of a first targeting nucleic acid construct and a second targeting nucleic acid construct,
wherein the first targeting nucleic acid construct comprises a nucleotide sequence of a human IL1RL2 gene, flanked by 5′ and 3′ mouse nucleotide sequences capable of mediating homologous recombination and integration of the nucleotide sequence of a human IL1RL2 gene into a mouse Il1rl2 gene to form a humanized Il1rl2 gene, wherein the humanized Il1rl2 gene encodes a humanized Il1rl2 protein that comprises the ectodomain of a human IL1RL2 protein and the transmembrane-cytoplasmic domains of a mouse Il1rl2 protein; and
wherein the second targeting nucleic acid construct comprises a contiguous nucleic acid sequence comprising a human IL1F6 gene, a human IL1F8 gene, and a human IL1F9 gene, wherein the contiguous nucleic acid sequence is flanked by 5′ and 3′ mouse nucleotide sequences capable of mediating homologous recombination and integration of the contiguous nucleic acid sequence into the mouse genomic DNA encompassing the mouse Il1f6 gene, the mouse Il1f8 gene, and the mouse Il1f9 gene.

47. The combination of claim 46, wherein the nucleotide sequence of a human IL1RL2 gene encodes the ectodomain of the human IL1RL2 protein.

48. The combination of claim 47, wherein the nucleotide sequence of a human IL1RL2 gene is a genomic fragment of the human IL1RL2 gene that comprises exons 3-8 of the human IL1RL2 gene.

49. A method of making a genetically modified mouse embryonic stem (ES) cell, comprising:
introducing the combination of claim 46 into mouse ES cells, and
selecting a mouse ES cell in which the nucleotide sequence of a human IL1RL2 gene in the first targeting nucleic acid construct has integrated into the mouse Il1rl2 gene to form a humanized Il1rl2 gene, and the contiguous nucleic acid sequence in the second targeting nucleic acid construct has integrated into the mouse genomic DNA encompassing the mouse Il1f6 gene, the mouse Il1f8 gene, and the mouse Il1f9 gene, thereby obtaining the genetically modified mouse ES cell.

50. The method of claim 49, wherein the nucleotide sequence of a human IL1RL2 gene encodes the ectodomain of the human IL1RL2 protein.

51. The method of claim 50, wherein the nucleotide sequence of a human IL1RL2 gene is a genomic fragment of the human IL1RL2 gene that comprises exons 3-8 of the human IL1RL2 gene.

52. A method of making a genetically modified mouse, comprising
(i) making a first mouse by
introducing the first targeting nucleic acid of the combination of claim 46 into mouse ES cells,
selecting a mouse ES cell in which the nucleotide sequence of the human IL1RL2 gene in the first targeting nucleic acid has replaced a genomic fragment of the endogenous mouse Il1rl2 gene to form the humanized Il1rl2 gene, and
making the first mouse from the selected mouse ES cell; and
(ii) making a second mouse by
introducing the second targeting nucleic acid of the combination of claim 46 into mouse ES cells,
selecting a mouse ES cell in which the contiguous nucleic acid sequence in the second targeting nucleic acid construct has replaced a mouse genomic fragment comprising the mouse Il1f6 gene, the mouse Il1f8 gene, and the mouse Il1f9 gene, and
making the second mouse from the selected mouse ES cell, and
(iii) crossing the first mouse with the second mouse to obtain the genetically modified mouse.

53. The method of claim 51, wherein the nucleotide sequence of a human IL1RL2 gene encodes the ectodomain of the human IL1RL2 protein.

54. The method of claim 53, wherein the nucleotide sequence of a human IL1RL2 gene is a genomic fragment of the human IL1RL2 gene that comprises exons 3-8 of the human IL1RL2 gene.


SEQUENCE LISTING (57 sequences)

SEQ ID NO: 1 - DNA, 2628 bp, Homo sapiens
  source 1..2628: mol_type = other DNA
cccgcccacggtggcggggaaatacctaggcatggaagtggcatgacagggctcgtgtccctgtcatattttccactctccacgaggtcctgcgcgcttcaatcctgcaggcagcccggtttggggatgtggtccttgctgctctgcgggttgtccatcgcccttccactgtctgtcacagcagatggatgcaaggacatttttatgaaaaatgagatactttcagcaagccagccttttgcttttaattgtacattccctcccataacatctggggaagtcagtgtaacatggtataaaaattctagcaaaatcccagtgtccaaaatcatacagtctagaattcaccaggacgagacttggattttgtttctccccatggaatggggggactcaggagtctaccaatgtgttataaagggtagagacagctgtcatagaatacatgtaaacctaactgtttttgaaaaacattggtgtgacacttccataggtggtttaccaaatttatcagatgagtacaagcaaatattacatcttggaaaagatgatagtctcacatgtcatctgcacttcccgaagagttgtgttttgggtccaataaagtggtataaggactgtaacgagattaaaggggagcggttcactgttttggaaaccaggcttttggtgagcaatgtctcggcagaggacagagggaactacgcgtgtcaagccatactgacacactcagggaagcagtacgaggttttaaatggcatcactgtgagcattacagaaagagctggatatggaggaagtgtccctaaaatcatttatccaaaaaatcattcaattgaagtacagcttggtaccactctgattgtggactgcaatgtaacagacaccaaggataatacaaatctacgatgctggagagtcaataacactttggtggatgattactatgatgaatccaaacgaatcagagaaggggtggaaacccatgtctcttttcgggaacataatttgtacacagtaaacatcaccttcttggaagtgaaaatggaagattatggccttcctttcatgtgccacgctggagtgtccacagcatacattatattacagctcccagctccggattttcgagcttacttgataggagggcttatcgccttggtggctgtggctgtgtctgttgtgtacatatacaacatttttaagatcgacattgttctttggtatcgaagtgccttccattctacagagaccatagtagatgggaagctgtatgacgcctatgtcttataccccaagccccacaaggaaagccagaggcatgccgtggatgccctggtgttgaatatcctgcccgaggtgttggagagacaatgtggatataagttgtttatattcggcagagatgaattccctggacaagccgtggccaatgtcatcgatgaaaacgttaagctgtgcaggaggctgattgtcattgtggtccccgaatcgctgggctttggcctgttgaagaacctgtcagaagaacaaatcgcggtctacagtgccctgatccaggacgggatgaaggttattctcattgagctggagaaaatcgaggactacacagtcatgccagagtcaattcagtacatcaaacagaagcatggtgccatccggtggcatggggacttcacggagcagtcacagtgtatgaagaccaagttttggaagacagtgagataccacatgccgcccagaaggtgtcggccgtttcctccggtccagctgctgcagcacacaccttgctaccgcaccgcaggcccagaactaggctcaagaagaaagaagtgtactctcacgactggctaagacttgctggactgacacctatggctggaagatgacttgttttgctccatgtctcctcattcctacacctattttctgctgcaggatgaggctagggttagcattctagacacccagttgagctcaggcgtagagaagaggaggatgggataagaactggggccatccccatgtcatggtgggtgagagctggggccatccccgtggtcatggagggtgagagctgggggttatccccatggtcatggagggtgagggctggtcgggggaggcatccccaagtcatggtgggtgagagctcggagcatccccatgtcatggtgggtgagatctgggggtatccctgtgtcatggtgggtgagggcgggtggtcatccacatggtcatagtgggtgagagctgggggtatccctacatcatggtgggtgagagctgggagcatccccatgtcatggtgggcgagatctggggggtatccccacgtcatggtggatgagagctgggggaatcaccatgtcatggtgggtgagatcttgggggatcacctgtcatggtgggtgagagctggggggatcacctgtcatggtgggtgagagttgggattcatccccatgtcatggtgggctgagcccacatggaagcctgtgcttggacagcgtatgcccttttctctgtttttccacaatgaacaattaaactgtaaatgttaaaaatatcagtaatttgtgaaataaattttattctcatttgagcaacataaaaaaaaaaaaaaaa

SEQ ID NO: 2 - protein, 575 aa, Homo sapiens
MWSLLLCGLSIALPLSVTADGCKDIFMKNEILSASQPFAFNCTFPPITSGEVSVTWYKNSSKIPVSKIIQSRIHQDETWILFLPMEWGDSGVYQCVIKGRDSCHRIHVNLTVFEKHWCDTSIGGLPNLSDEYKQILHLGKDDSLTCHLHFPKSCVLGPIKWYKDCNEIKGERFTVLETRLLVSNVSAEDRGNYACQAILTHSGKQYEVLNGITVSITERAGYGGSVPKIIYPKNHSIEVQLGTTLIVDCNVTDTKDNTNLRCWRVNNTLVDDYYDESKRIREGVETHVSFREHNLYTVNITFLEVKMEDYGLPFMCHAGVSTAYIILQLPAPDFRAYLIGGLIALVAVAVSVVYIYNIFKIDIVLWYRSAFHSTETIVDGKLYDAYVLYPKPHKESQRHAVDALVLNILPEVLERQCGYKLFIFGRDEFPGQAVANVIDENVKLCRRLIVIVVPESLGFGLLKNLSEEQIAVYSALIQDGMKVILIELEKIEDYTVMPESIQYIKQKHGAIRWHGDFTEQSQCMKTKFWKTVRYHMPPRRCRPFPPVQLLQHTPCYRTAGPELGSRRKKCTLTTG

SEQ ID NO: 3 - DNA, 4072 bp, Mus musculus
  source 1..4072: mol_type = other DNA
ggcggacagagaccggaagctgggagatgatggcttctgatgtttgttgcttacgcgacacagcttgtttggaattatgtggcccttgacaactgcgtggcacacacttcaagatacaaaagtgagaagaagaggggggtcaccagaggagactgaagtcagtaagtttctaccaaagctgggtttcttctggccactccaaacaaagctgaaattgtccttcagacctctcctgagatgggggttacatctttgctcttctgtggggtgtttttcctgcttctgcttttcgtggcagcagatacgtgtgaggacatttttatgcacaatgtgataatttcagagggccagccttttcctttcaactgcacatacccgccagaaacaaacggggcagtaaatctgacatggtacaaaacacctagcaaaagcccagtatctaacaacagacaccttagagttcaccaggaccagacctggatcttgtttcttccattgacactggaggactccggtatctatcagtgtgttataaggaatgcccacaactgctaccaaatagctgtgaacctaaccgttttaaaaaaccactggtgtgactcttccatggaggggagtcccgtaaattcaccagatgtgtaccagcaaatattacccataggaaaatcgggcagtctgaattgtcatctctacttcccagaaagttgtgctttggattcaataaaatggtataagggttgtgaagagattaaagcggggaaaaagtacagcccttcaggagcaaagcttcttgtgaacaacgttgctgtggaggacggcgggagctatgcgtgctcagccagactgactcacttggggagacacttcaccattagaaactacattgctgtgaacaccaaggaagttgagtatggaagaaggatccctaacatcacgtatccaaagaacaactccattgaagttccacttggctccaccctcatcgtgaactgcaatataacagacacgaaggagaatacaaacctgaggtgctggagagtcaacaacaccctggtggatgactactacaaagactccaaacgcatccaggaaggaatcgaaaccaatgtgtccttgagggatcaaattcggtacacagtgaacataacattcttaaaagtgaaaatggaggactacggccgtcctttcacgtgtcatgctggagtgtccgcagcctacatcattctgatatacccagttccagacttcagggcttacctcttaggagggcttatggcgttcctacttctggttgtatctgttctgttcatctacaacagctttaagatcgacatcatgctttggtacagaagcgccttccacactgcccaggctccagatgatgagaagctgtatgatgcctatgtcttataccccaagtacccgagaggaagccagggccatgatgtagacacactggtactgaagatcttgcccgaggtgctggagaaacagtgtggatataagttatttatatttggcagggatgaattccctggacaagctgtggccagcgtcattgatgaaaacattaagctgtgtaggaggctgatggtctttgtggcaccagagtcgtctagcttcggctttctaaagaacttgtcagaagaacaaattgccgtctacaatgccctcatccagcatggcatgaaggtcattctgatcgaactggagaaagtcaaagactacagcaccatgcccgagtccattcagtatatccgacagaagcacggggccatccaatgggatggggacttcacagagcagtcacagtgtgccaagaccaagttctggaagaaagtgagataccatatgccacccaggaggtatccagcatcttccccagtccagctgctgggacacataccctgcaattgcaaggcaggcaaatgcaatgctgccacagggctcataactccctgagagtggttagtgtgtgttggctcacactacatcctctctgaattgtctactcatgtagctggctcttttgtgcttgtgagcgaccttgtccttgcccttgtagctttttgtttatttgtattgtttgctggatgctttcagtcatagctgatccttattactcctgtttgcttcatggctcctgaaaacccatatactccccaagcatggggtggctctaacgttgggcctccgtgtcagtgggatactgaggatgacagatcaggtgtccaggatgcttgatcctggtttgagatgggaggctgggcagtctgaggataccaagtagatagccttagggaagatcagcaaggacagtagtgcaatgtctgtgtttgtccagggtccacaagattctagtcctcaggcagtgtagactgcatttgaatctccttcattttcctattgcatttagttaactaaccattgtgagaacaggacagaggactgggagttgggggtggggggaggtgacagaggatgggagtgtgtgtgtaagaggtgacagttacatgatcaatcctgggaaatcagactcaggaaaccagcattagggtgcagatctgagttttttgtgcagcccttaagcttatatacagcgttcgagccaatcccaagcccctaaaccctcggccaatcatatcttgccacatcatcactatgcctcaatgaaagccctgcctgatgaggtaactcaaaaggaattggggtgggaagcatcatcccctgtcagaacttccgtgaagatggaggacgtagccactaccccacctcagtttctttttgttgtctcaggtttgcaccagagccatcttaaatctagtgacatgtgcagcaaatcatgactcttcaaggagctgcgggagcctgtggtgcctcgctctccatcccactttttccttcacagggttgacttccacgtcccccaaaccatggtattagcacgatgcccctagaagccgcttagatgtcgccaggaccaatggtcttcatttccctctttcccagcccctgtgaatcttactattctaggaaattcaaatgagagacacggtgcattcacggtttctcattttatgactggcttctttcatacaccacggatttctcaagtgctctcgagattcatccattgattagccttttgttttattattatcactactgtctttctcttctttttcttcctctttaaatttctccccctcctcacgagcatccggagaggagcatagactctctgcatgcctacagcgtgatgagctcaccgagggtcccttcaatcacaagcttgtatgttttgcttttcggtttctctttgcaccatgggaattgtaactccttacagaggatttgaaggttcgggtacaaaggcgtcactgactggttccttcttctgtcttcctcaccctgaatgggctttgcttctgggtgattcctggaaattgtcagcagtgatggagaaacctagggacagggctgactaatggggcctggcctttcttctttgaagcccaggacacagtgcctgcctagtgggtcctggacacaccaggtggttccactctgctcagtggtaacattttttagctgagtcatcaacatcgggtcctttagtgaggaggtgcgggcatctctccccagttagatttggactcttacttccctgctcatttccctttgaaagattcaggagactatgtcagaagcagggagccagcaccctgtgtcttctccacgtggctccttgtcacacctgtgaccaccaattttagaaaatgtgtttccttgttctgaccacacttatgcggacattagttgaggctgttgtcagaaaggaaaattaactcatttgaagtaaacattttctggaaatctctgacttctgtcaggccaatggtgctgaaaaatttaaaactgaattcacttttttggccagaaagctttcctcttgccaacatgtcttaattttccttttgcttaaacttattggagatgtattatgaatgctaagatgtgagtgtgttatatcagtacattgacattttaaataaagtatattttaataaaa

SEQ ID NO: 4 - protein, 574 aa, Mus musculus
MGVTSLLFCGVFFLLLLFVAADTCEDIFMHNVIISEGQPFPFNCTYPPETNGAVNLTWYKTPSKSPVSNNRHLRVHQDQTWILFLPLTLEDSGIYQCVIRNAHNCYQIAVNLTVLKNHWCDSSMEGSPVNSPDVYQQILPIGKSGSLNCHLYFPESCALDSIKWYKGCEEIKAGKKYSPSGAKLLVNNVAVEDGGSYACSARLTHLGRHFTIRNYIAVNTKEVEYGRRIPNITYPKNNSIEVPLGSTLIVNCNITDTKENTNLRCWRVNNTLVDDYYKDSKRIQEGIETNVSLRDQIRYTVNITFLKVKMEDYGRPFTCHAGVSAAYIILIYPVPDFRAYLLGGLMAFLLLVVSVLFIYNSFKIDIMLWYRSAFHTAQAPDDEKLYDAYVLYPKYPRGSQGHDVDTLVLKILPEVLEKQCGYKLFIFGRDEFPGQAVASVIDENIKLCRRLMVFVAPESSSFGFLKNLSEEQIAVYNALIQHGMKVILIELEKVKDYSTMPESIQYIRQKHGAIQWDGDFTEQSQCAKTKFWKKVRYHMPPRRYPASSPVQLLGHIPCNCKAGKCNAATGLITP

SEQ ID NO: 5 - DNA, 2044 bp, Rattus norvegicus
  source 1..2044: mol_type = other DNA
ccggctggcctaggatcaggcaagaaaaggctgaacgcctttctaaggacggactctttctgtacagctccacttggggaagcccgaaatggggatgccacccttgctcttctgttgggtgtctttcgtgcttccactttttgtggcagcaggtaactgtactgatgtctatatgcaccatgagatgatttcagagggccagcctttccccttcaactgcacataccctccagtaacaaacggggcagtgaatctgacatggcatagaacacccagtaagagcccaatctccatcaacagacacgttagaattcaccaggaccagtcctggattttgtttcttccgttggcattggaggactcaggcatctatcaatgtgttataaaggatgcccacagctgttaccgaatagctataaacctaaccgtttttagaaaacactggtgcgactcttccaacgaagagagttccataaattcctcagatgagtaccagcaatggttacccataggaaaatcgggcagtctgacgtgccatctctacttcccagagagctgtgttttggattcaataaagtggtataagggttgtgaagagattaaagtgagcaagaagttttgccctacaggaacaaagcttcttgttaacaacatcgacgtggaggatagtgggagctatgcatgctcagccagactgacacacttggggagaatcttcacggttagaaactacattgctgtgaataccaaggaagttgggtctggaggaaggatccctaacatcacgtatccaaaaaacaactccattgaagttcaacttggctccaccctcattgtggactgcaatataacagacacgaaggagaatacgaacctcagatgctggcgagttaacaacaccctggtggacgattactacaacgacttcaaacgcatccaggaaggaatcgaaaccaatctgtctctgaggaatcacattctgtacacagtgaacataacattcttagaagtgaaaatggaggactacggccatcctttcacatgccacgctgcggtgtccgcagcctacatcattctgaaacgcccagctccagacttccgggcttacctcataggaggtctcatggctttcctacttctggccgtgtccattctgtacatctacaacacctttaaggtcgacatcgtgctttggtataggagtaccttccacactgcccaggctccagatgacgagaagctgtatgatgcctatgtcttataccccaagtacccaagagaaagccagggccatgatgtggacacactggtgttgaagatcttgcccgaggtgctggagaaacagtgtggatataagttattcatatttggcagggatgaattccctggacaagctgtggccagcgtcattgatgaaaacattaagctgtgtaggaggctgatggtcctcgtggcaccagagacatccagcttcagctttctgaagaacttgactgaagaacaaatcgctgtctacaatgccctcgtccaggacggcatgaaggtcattctgattgaactggagagagtcaaggactacagcaccatgcccgagtccattcagtacatccgacagaagcacggggccatccagtgggatggggacttcacagagcaggcacagtgcgccaagacgaaattctggaagaaagtgagatatcatatgccacccaggaggtacccggcatctccccccgtccagctgctaggacacacaccccgcataccaggctagtgcagtgccaccgccacggggctcataactccttaagagcggttagtgtgtggtggctcgcactacaacctctctggatcatctacccccgtagcttgctcttttgtgcttgtgagcgacctcgtccttagccacgtcatattttgattttttgtttgttttgtttgtttgttgtatgcttttagtcatagctgattcgtactactcctgtttgcttcatggttcctgaatcccagagactccctgagcatgggtggctatcatgttggg

SEQ ID NO: 6 - protein, 561 aa, Rattus norvegicus
MGMPPLLFCWVSFVLPLFVAAGNCTDVYMHHEMISEGQPFPFNCTYPPVTNGAVNLTWHRTPSKSPISINRHVRIHQDQSWILFLPLALEDSGIYQCVIKDAHSCYRIAINLTVFRKHWCDSSNEESSINSSDEYQQWLPIGKSGSLTCHLYFPESCVLDSIKWYKGCEEIKVSKKFCPTGTKLLVNNIDVEDSGSYACSARLTHLGRIFTVRNYIAVNTKEVGSGGRIPNITYPKNNSIEVQLGSTLIVDCNITDTKENTNLRCWRVNNTLVDDYYNDFKRIQEGIETNLSLRNHILYTVNITFLEVKMEDYGHPFTCHAAVSAAYIILKRPAPDFRAYLIGGLMAFLLLAVSILYIYNTFKVDIVLWYRSTFHTAQAPDDEKLYDAYVLYPKYPRESQGHDVDTLVLKILPEVLEKQCGYKLFIFGRDEFPGQAVASVIDENIKLCRRLMVLVAPETSSFSFLKNLTEEQIAVYNALVQDGMKVILIELERVKDYSTMPESIQYIRQKHGAIQWDGDFTEQAQCAKTKFWKKVRYHMPPRRYPASPPVQLLGHTPRIPG

SEQ ID NO: 7 - protein, 573 aa, synthetic construct
  REGION 1..573: note = Humanized Il1r12
MGVTSLLFCGVFFLLLLFVAADGCKDIFMKNEILSASQPFAFNCTFPPITSGEVSVTWYKNSSKIPVSKIIQSRIHQDETWILFLPMEWGDSGVYQCVIKGRDSCHRIHVNLTVFEKHWCDTSIGGLPNLSDEYKQILHLGKDDSLTCHLHFPKSCVLGPIKWYKDCNEIKGERFTVLETRLLVSNVSAEDRGNYACQAILTHSGKQYEVLNGITVSITERAGYGGSVPKIIYPKNHSIEVQLGTTLIVDCNVTDTKDNTNLRCWRVNNTLVDDYYDESKRIREGVETHVSFREHNLYTVNITFLEVKMEDYGLPFMCHAGVSTAYIILQLPVPDFRAYLLGGLMAFLLLVVSVLFIYNSFKIDIMLWYRSAFHTAQAPDDEKLYDAYVLYPKYPRGSQGHDVDTLVLKILPEVLEKQCGYKLFIFGRDEFPGQAVASVIDENIKLCRRLMVFVAPESSSFGFLKNLSEEQIAVYNALIQHGMKVILIELEKVKDYSTMPESIQYIRQKHGAIQWDGDFTEQSQCAKTKFWKKVRYHMPPRRYPASSPVQLLGHIPCNCKAGKCNAATGLITP

SEQ ID NO: 8 - DNA, 888 bp, Homo sapiens
  source 1..888: mol_type = other DNA
aaaacccaagtgcagtagaagccattgttcataatggtagggatacagggtccttcgtaacagattatcagtgtggcctatgctggaaagtctggtgacctctgattttttttgcttccaggtctttggccttggcactctttgtcatattagagttcctgggtctaggcctgggcaggattcataggtgcagctgcttctgctggaggtagactgcatccaacaaagtaagggtgctgggtgagttctgggagtatagattctgactggggtcactgctgggctggccgccagtctttcatctgacccagggttaaactgtggcttgggactgactcaggtcctctcttggggtcggtctgcacataaaaggactcctatccttggcagttctgaaacaacaccaccacaatggaaaaagcattgaaaattgacacacctcagcaggggagcattcaggatatcaatcatcgggtgtgggttcttcaggaccagacgctcatagcagtcccgaggaaggaccgtatgtctccagtcactattgccttaatctcatgccgacatgtggagacccttgagaaagacagagggaaccccatctacctgggcctgaatggactcaatctctgcctgatgtgtgctaaagtcggggaccagcccacactgcagctgaaggaaaaggatataatggatttgtacaaccaacccgagcctgtgaagtcctttctcttctaccacagccagagtggcaggaactccaccttcgagtctgtggctttccctggctggttcatcgctgtcagctctgaaggaggctgtcctctcatccttacccaagaactggggaaagccaacactactgactttgggttaactatgctgttttaa

SEQ ID NO: 9 - protein, 158 aa, Homo sapiens
MEKALKIDTPQQGSIQDINHRVWVLQDQTLIAVPRKDRMSPVTIALISCRHVETLEKDRGNPIYLGLNGLNLCLMCAKVGDQPTLQLKEKDIMDLYNQPEPVKSFLFYHSQSGRNSTFESVAFPGWFIAVSSEGGCPLILTQELGKANTTDFGLTMLF

SEQ ID NO: 10 - DNA, 883 bp, Mus musculus
  source 1..883: mol_type = other DNA
taacctcctcttttcgtgttgtggataaaaggcttggaccttagtagagcttgtcagagaagctgagcacagtagtgggtgtagttctgtagtgtgcagacacattcctattcaatcagggtcaatctgcagattggcagctcagaaacaacatcaccataatgaataaggagaaagaactaagagcagcatcaccttcgcttagacatgttcaggatcttagtagtcgtgtgtggatcctgcagaacaatatcctcactgcagtcccaaggaaagagcaaacagttccagtcactattaccttgctcccatgccaatatctggacactcttgagacgaacaggggggatcccacgtacatgggagtgcaaaggccgatgagctgcctgttctgcacaaaggatggggagcagcctgtgctacagcttggggaagggaacataatggaaatgtacaacaaaaaggaacctgtaaaagcctctctcttctatcacaagaagagtggtacaacctctacatttgagtctgcagccttccctggttggttcatcgctgtctgctctaaagggagctgcccactcattctgacccaagaactgggggaaatcttcatcactgacttcgagatgattgtggtacattaaggtttttagacacattgctctgtggcactctctcaagatttcttggattctaacaagaagcaatcaaagacacccctaacaaaatggaagactgaaaagaaagctgagccctccctgggctgtttttccttggtggtgaatcagatgcagaacatcttaccatgttttcatccaaagcatttactgttggtttttacaaggagtgaattttttaaaataaaatcatttatctcatttgc

SEQ ID NO: 11 - protein, 160 aa, Mus musculus
MNKEKELRAASPSLRHVQDLSSRVWILQNNILTAVPRKEQTVPVTITLLPCQYLDTLETNRGDPTYMGVQRPMSCLFCTKDGEQPVLQLGEGNIMEMYNKKEPVKASLFYHKKSGTTSTFESAAFPGWFIAVCSKGSCPLILTQELGEIFITDFEMIVVH

SEQ ID NO: 12 - DNA, 869 bp, Rattus norvegicus
  source 1..869: mol_type = other DNA
taatctcctcccttcatgttttggataaaaggcctggaccctagcagagcttgtcagagaagctgagcacagtagtggctgtagttctgtagtgtgcagatacattcctctgcaatcaggatcagtctgcagattggcagttcagaaacaacatcaccataatgaataaggataaagaactaagtgcagcaacaccttggtttagacatattcaggatcttagtagtcgtgtgtgggtccttcaagacaatattcttactgcagtcccaaggaaagagcaaacggttccagtcactatcaccttactcccatgccaatatctggacactcttgagaagaacaaaggggatcccatgtacctgggagtgaagaaacctcaaagttgtctgtcctgcacaaagaatggggagcagcctgtactacaacttcgggaagggaacatactggatatgtaccaccagaaggaacgtgtaaaagcctctctcttctatcacaagaagagtggtacaacctctacattcgagtctgcagccttccctggttggttcattgctgtctgctccaaagggagctgcccactcgttttgacccaagaacttgggaaaaccttcatcactgattttgagatgactgtagtacattaagattgctctgcagcactctctgaagatctctcggattctaacaagcaaccaaacacacccccaacaaaatacaagactgaaaagaaagctgagccctccctgggcttttttcccttggcaggtggatcagatgcagaacttcttaccatgttttcatccaaagcatttactgatggtttttacaagaagaaaacgtttttaattaaaatcatttatctcacttgc

SEQ ID NO: 13 - protein, 160 aa, Rattus norvegicus
MNKDKELSAATPWFRHIQDLSSRVWVLQDNILTAVPRKEQTVPVTITLLPCQYLDTLEKNKGDPMYLGVKKPQSCLSCTKNGEQPVLQLREGNILDMYHQKERVKASLFYHKKSGTTSTFESAAFPGWFIAVCSKGSCPLVLTQELGKTFITDFEMTVVH

SEQ ID NO: 14 - DNA, 1186 bp, Homo sapiens
  source 1..1186: mol_type = other DNA
cacgggttcctccccactctgtctttctcacctctccttcacttttcctagcctcctcaccaccatctgatctatcttgttctcttcacaaaaggctctgaagacatcatgaacccacaacgggaggcagcacccaaatcctatgctattcgtgattctcgacagatggtgtgggtcctgagtggaaattctttaatagcagctcctcttagccgcagcattaagcctgtcactcttcatttaatagcctgtagagacacagaattcagtgacaaggaaaagggtaatatggtttacctgggaatcaagggaaaagatctctgtctcttctgtgcagaaattcagggcaagcctactttgcagcttaagcttcagggctcccaagataacatagggaaggacacttgctggaaactagttggaattcacacatgcataaacctggatgtgagagagagctgcttcatgggaacccttgaccaatggggaataggagtgggtagaaagaagtggaagagttcctttcaacatcaccatctcaggaagaaggacaaagatttctcatccatgcggaccaacataggaatgccaggaaggatgtagaaataaggggaggaagattcccatctctacaatctttgagtgggtttgctatcaatgaaatgctacaaatggaataagttgcagaaatttttctcttttcttgggttctggagagtttgtaaaacaaggacactatgtatttttaaagagttggtaaatcttacctgtaaagctagagaaggtcggagtctttttaggagtagatttggactacataacctgtaaatgtgttttgtccagtccttagagtgttttttaaaaaattgtaaagtcaaggttttcatgaaaaatgggaagatcagacaacattgctcctgaattcccacagagcagcaagctactagagctcaatctgttatttcttttcctgatgtacaggggttaagtcctatggaagaaacagcagaattattcaaaattatttacataatgtgcaattattcactagagcatgaggagtgaaacgctctgtttagtatgtataacttaaaaggaacacatacaattaaaagtaattgaaagacatttcttcttaaaaattctataatcttacactggtaaaataaactagtttttcccatgt

SEQ ID NO: 15 - protein, 164 aa, Homo sapiens
MNPQREAAPKSYAIRDSRQMVWVLSGNSLIAAPLSRSIKPVTLHLIACRDTEFSDKEKGNMVYLGIKGKDLCLFCAEIQGKPTLQLKLQGSQDNIGKDTCWKLVGIHTCINLDVRESCFMGTLDQWGIGVGRKKWKSSFQHHHLRKKDKDFSSMRTNIGMPGRM

SEQ ID NO: 16 - DNA, 790 bp, Mus musculus
  source 1..790: mol_type = other DNA
gactggcctcattgcctgctgtcataacttcgatcccagagacaagacttctttggaccaaggcaatgatggctttccctccacaatcttgtgtacatgttcttcctccaaagagtattcaaatgtgggaaccgaatcataacactatgcatggatcctcacaatctcccagaaactacagggttcatgactcacaacagatggtatgggtcctgactggaaatactttaacagcagttcctgctagcaacaatgtcaagcctgtcattcttagcttgatagcatgtagagacacggaattccaagatgtaaagaaaggtaatctagttttcctgggaatcaagaacagaaatctctgcttctgctgtgttgagatggagggcaaaccaactttgcagcttaaggaagtagacatcatgaatttgtacaaagagagaaaagcacaaaaagcctttctgttctatcatggcatagagggctccacttctgtctttcagtcagtcctctatcctggctggtttatagccacctcttccatagaaagacagacaatcatcctcacacatcagcggggtaaattggttaacactaacttctacatagagtctgagaagtaaatccaacatgggtcatatgtggccagctccaggccacagaatcaaactgttgaagaatcttctacttgaaaacatagcaagcctcttagaagaccaccatgccatgtcttcaccagacatccatgtctaaaatgccactcatttgaatatcattccagcatgaagcccatgaa

SEQ ID NO: 17 - protein, 183 aa, Mus musculus
MMAFPPQSCVHVLPPKSIQMWEPNHNTMHGSSQSPRNYRVHDSQQMVWVLTGNTLTAVPASNNVKPVILSLIACRDTEFQDVKKGNLVFLGIKNRNLCFCCVEMEGKPTLQLKEVDIMNLYKERKAQKAFLFYHGIEGSTSVFQSVLYPGWFIATSSIERQTIILTHQRGKLVNTNFYIESEK

SEQ ID NO: 18 - DNA, 587 bp, Rattus norvegicus
  source 1..587: mol_type = other DNA
ggcgtgggtcctggctggaaattctttgacagcggttcctgctagcaacagcgtcaaatctgtcattcttagcttgatagcatgtagagacatggaattccaagatgaaaagaagggtaatctagttttcctgggaatcaaaggcagaagtctctgcctcttctgtgctgagatcgagggcaaaccgactttgcagcttaaggatgtagacatcatggatttgtacaatgagaaaaaagcacagaaagcctttctcttctaccatggcatagagggatctacttctgtctttcagtcagtcttgtatcctggctggtttatagccacctcttccacagcaagacaaacaatcattcttacacaggagaggggtgaagctaataacactaacttctacttagagtctgagaattagatctaccatgggccatatgtggccagttgcaggccaaagaatcaaactgttggagaatcttttacttgaaaacatagcaagacatcttagaagaccaccatgtttcctcttctcctgacatccatgtctgaaatgccattcattggtataagagaataaaaaatagggctgg

SEQ ID NO: 19 - protein, 179 aa, Rattus norvegicus
MMAFPPQSHVCFTPPKSSDFDNINKGVQAFPSTYRVHDSQHKAWVLAGNSLTAVPASNSVKSVILSLIACRDMEFQDEKKGNLVFLGIKGRSLCLFCAEIEGKPTLQLKDVDIMDLYNEKKAQKAFLFYHGIEGSTSVFQSVLYPGWFIATSSTARQTIILTQERGEANNTNFYLESEN

SEQ ID NO: 20 - DNA, 1212 bp, Homo sapiens
  source 1..1212: mol_type = other DNA
gaagctgctggagccacgattcagtcccctggactgtagataaagaccctttcttgccaggtgctgagacaaccacactatgagaggcactccaggagacgctgatggtggaggaagggccgtctatcaatcaatgtgtaaacctattactgggactattaatgatttgaatcagcaagtgtggacccttcagggtcagaaccttgtggcagttccacgaagtgacagtgtgaccccagtcactgttgctgttatcacatgcaagtatccagaggctcttgagcaaggcagaggggatcccatttatttgggaatccagaatccagaaatgtgtttgtattgtgagaaggttggagaacagcccacattgcagctaaaagagcagaagatcatggatctgtatggccaacccgagcccgtgaaacccttccttttctaccgtgccaagactggtaggacctccacccttgagtctgtggccttcccggactggttcattgcctcctccaagagagaccagcccatcattctgacttcagaacttgggaagtcatacaacactgcctttgaattaaatataaatgactgaactcagcctagaggtggcagcttggtctttgtcttaaagtttctggttcccaatgtgttttcgtctacattttcttagtgtcattttcacgctggtgctgagacaggggcaaggctgctgttatcatctcattttataatgaagaagaagcaattacttcatagcaactgaagaacaggatgtggcctcagaagcaggagagctgggtggtataaggctgtcctctcaagctggtgctgtgtaggccacaaggcatctgcatgagtgactttaagactcaaagaccaaacactgagctttcttctaggggtgggtatgaagatgcttcagagctcatgcgcgttacccacgatggcatgactagcacagagctgatctctgtttctgttttgctttattccctcttgggatgatatcatccagtctttatatgttgccaatatacctcattgtgtgtaatagaaccttcttagcattaagaccttgtaaacaaaaataattcttgtgttaagttaaatcatttttgtcctaattgtaatgtgtaatcttaaagttaaataaactttgtgtatttatataataataaagctaaaactgatataaaataaagaaagagtaaactg

SEQ ID NO: 21 - protein, 169 aa, Homo sapiens
MRGTPGDADGGGRAVYQSMCKPITGTINDLNQQVWTLQGQNLVAVPRSDSVTPVTVAVITCKYPEALEQGRGDPIYLGIQNPEMCLYCEKVGEQPTLQLKEQKIMDLYGQPEPVKPFLFYRAKTGRTSTLESVAFPDWFIASSKRDQPIILTSELGKSYNTAFELNIND

SEQ ID NO: 22 - DNA, 1647 bp, Mus musculus
  source 1..1647: mol_type = other DNA
atcattacacctgggagctgcccaagctgggatttggtcttctagactttccataaggactctttcttgccatgtacctacaaacctacaaaaccctttgagtgcgcctctgagtgtggagctttctgcagttcagggttcagtgaatggacagatctgtcttgctgtaagtgatcctgtgagcgatcctgtgagcgatcctgtgagcgatcctgtgagcgatcctgtgagcgatcctgtgagtgatcctgtgagcaatcctgtgagagctctgtgcaccagaacaagatcacgatggaaaacaatgaaaaaaaaaacattgtgtatggaagtgatgttgagatggaacacgagagagctgggctatttgtatcttcagctatgttttctaaacacccattttctacacacatctcaggaagagaaactcctgactttggggaggtttttgacttggaccagcaggtgtggatctttcgtaatcaggcccttgtgacagttccacgaagccacagagtaaccccagtcagcgtgactatcctcccatgcaagtacccagagtctcttgaacaggacaaagggattgccatttatttgggaattcagaatccagataaatgcctgttttgtaaggaagttaatggacaccctactttgctgctaaaggaagagaagattttggatttgtaccaccaccctgagccaatgaagccattcctgttttaccacacccggacaggtggaacatccacctttgaatcagtggctttccctggccactatattgcctcctccaagactggcaaccccatcttcctcacatcaaaaaagggagaatattacaacattaacttcaatttagatataaagtcttaaactcagcatggaagtggagggttggttagaactcttcctcttaaaacatcaaacctctaatatgttatcatttgtttgatctatggtattttcatactgaaaggtaagcaggaccaacattatctcctttcatagatgaagaagcaacaaaataatgcatactccaaagtcggtgggattggagtggtgtaaggatgttctctaagactgaagtggtccaatctataagacatcatgtctgagaaatagggtgctgtgccacagtgtataaaacctagaatgccctgtgtgtgagtggtgacatcctcatttccagccattggcctgattcataccatgttttcacacatttgtaatatcaactccttgcatttggatagcagaggcctcacaatgacatttgcctcaacagctaatagctttgatctttcaatgctgtgtagatctgaatacactagaaaaggaattagaatcctgagtgaatgaactgtgaggagccgccctcacatttgccattataagatggcactgacagctgtgttctaagtggtaaacatagtctgcacacatgcaggggcagttttcccaccatgtgttctgcctttcccgtgatgacaactgggccgatgggctgcagccaatcagggagtaatacgtcctaggtggaggataattctccttaaaagggacggggttctggcactctctcccttccttgcttgctctcttgcactctggctcctgaagatgtaagcaataaagctttgccgc

SEQ ID NO: 23 - protein, 164 aa, Mus musculus
MFSKHPFSTHISGRETPDFGEVFDLDQQVWIFRNQALVTVPRSHRVTPVSVTILPCKYPESLEQDKGIAIYLGIQNPDKCLFCKEVNGHPTLLLKEEKILDLYHHPEPMKPFLFYHTRTGGTSTFESVAFPGHYIASSKTGNPIFLTSKKGEYYNINFNLDIKS

SEQ ID NO: 24 - DNA, 1395 bp, Rattus norvegicus
  source 1..1395: mol_type = other DNA
ggaagctgcccaagctgggatttgatcttctgaactttccataaggactctttcttgccaggcaccagaacaagctcatgatggaaaacaatgaaaaaaatcgttacatatgggtgtgatgttgagatgtgacactaacgaactgggctatttgtatcttcagctatgtcctctaaatacccacattctccatgtactgcctcagcaggaaaagaaactcctgaccttgggcaggtttctgatgtggatcagcaggtgtggatctttcgtgatcaggcccttgtgacagttccacgaagccacactgtaactccagtcactgtgactgtcctcccatgcaagtacccagagtctcttgagcagggcaaagggactcccatttatttgggaattcaaaatccagataaatgcctgttttgtaaggaagttaatggacaccccactttgctcctgaaggaagagaagattttgaatttgtaccaccatcctgagccaatgaagccattcctgttttaccacaccctgacaggtgcaacgtccacctttgaatcagtggttttccctggcagctttattgcctcctccaagattggcaaacccatcttcctcacatcaaaaaagggagaacattacaacattcacttcagtttagatataatttagatataaagtcttgaactcagaatggaggtggagggttggttagaactcttataacttcaaacctctaatatgctatcatttgtttgatgtgtggttgtttcatactgagaagtgagcaaaaccaacattatctcatttcatagatgaagaagcaacaaaacagaatgtgtactccaaagtaggttggatgggagtggtgtaaggctctcctctaagactgaagtggtccaacccataaggcatcatgcctttctcaggtgctattatagggtgctgtgccacagtatataaaaactagaatgccccatgtgtgagtagcaacatcctcacttccagtcattggcctgattcataccaggttttcacacacttgtaataccaattccttgcatttggatagcaggagcctcacaatgacatttgctgccaacagctgatagctttgatccttcagcaactgactgctacgtggatctgaagacgccagaaaaggaattagaatcctaagtgaacgaacagtttttaaaatgtctagaactatttaaactatgttagaatacagtggtttatccttgaaaggtaaggatactgcctggtaaatcaaaaacagttaggtcaataaactcagcttgaacaattctttcctggaaacaatgtgtacaagtaatgattaaaacattaccttttattattctagacttccataaaaaaaaaaaaaaaaaaaaaaaaaaa

SEQ ID NO: 25 - protein, 165 aa, Rattus norvegicus
MSSKYPHSPCTASAGKETPDLGQVSDVDQQVWIFRDQALVTVPRSHTVTPVTVTVLPCKYPESLEQGKGTPIYLGIQNPDKCLFCKEVNGHPTLLLKEEKILNLYHHPEPMKPFLFYHTLTGATSTFESVVFPGSFIASSKIGKPIFLTSKKGEHYNIHFSLDII

SEQ ID NO: 26 - DNA, 108 bp, synthetic construct
  misc_feature 1..108: note = Synthetic Oligonucleotide
  source 1..108: mol_type = other DNA
tacatttgaaggtcttagcattatgcttctaaatgactgggctagtttgggcgatcgcgacaagaagggttgatttactctgtaggtgagtacagagtaaagaactct

SEQ ID NO: 27 - DNA, 140 bp, synthetic construct
  misc_feature 1..140: note = Synthetic Oligonucleotide
  source 1..140: mol_type = other DNA
caccctgagcacatggggaatccttcttgggttgcaacatgtggaaggacctcgagataacttcgtataatgtatgctatacgaagttatatgcatggcctccgcgccgggttttggcgcctcccgcgggcgcccccctc

SEQ ID NO: 28 - DNA, 181 bp, synthetic construct
  misc_feature 1..181: note = Synthetic Oligonucleotide
  source 1..181: mol_type = other DNA
tttcactgcattctagttgtggtttgtccaaactcatcaatgtatcttatcatgtctggaataacttcgtataatgtatgctatacgaagttatgctagtaactataacggtcctaaggtagcgagctagccagaatcaatgacaaagatgaagaatatatttaacctcataaacttggtt

SEQ ID NO: 29 - DNA, 177 bp, synthetic construct
  misc_feature 1..177: note = Synthetic Oligonucleotide
  source 1..177: mol_type = other DNA
caccctgagcacatggggaatccttcttgggttgcaacatgtggaaggacctcgagataacttcgtataatgtatgctatacgaagttatgctagtaactataacggtcctaaggtagcgagctagccagaatcaatgacaaagatgaagaatatatttaacctcataaacttggtt

SEQ ID NO: 30 - DNA, 100 bp, synthetic construct
  misc_feature 1..100: note = Synthetic Oligonucleotide
  source 1..100: mol_type = other DNA
ctggctctgtcaactagattggaaggcatgcggagctgccagtctcaactcgtcgacgtcactctcctttcccactgcatgtgaagcatttgagtgtctg

SEQ ID NO: 31 - DNA, 140 bp, synthetic construct
  misc_feature 1..140: note = Synthetic Oligonucleotide
  source 1..140: mol_type = other DNA
tgcactcagaaaagacaagtattcacattttttcttgtggctgatctggactcgagataacttcgtataatgtatgctatacgaagttatatgcatggcctccgcgccgggttttggcgcctcccgcgggcgcccccctc

SEQ ID NO: 32 - DNA, 181 bp, synthetic construct
  misc_feature 1..181: note = Synthetic Oligonucleotide
  source 1..181: mol_type = other DNA
tttcactgcattctagttgtggtttgtccaaactcatcaatgtatcttatcatgtctggaataacttcgtataatgtatgctatacgaagttatgctagtaactataacggtcctaaggtagcgagctagcctatgcctgcacagcccttccataggtactaaggatttaaactctcgttt

SEQ ID NO: 33 - DNA, 177 bp, synthetic construct
  misc_feature 1..177: note = Synthetic Oligonucleotide
  source 1..177: mol_type = other DNA
tgcactcagaaaagacaagtattcacattttttcttgtggctgatctggagtcgagataacttcgtataatgtatgctatacgaagttatgctagtaactataacggtcctaaggtagcgagctagcctatgcctgcacagcccttccataggtactaaggatttaaactctcgttt

SEQ ID NO: 34 - DNA, 24 bp, synthetic construct
  misc_feature 1..24: note = Synthetic Oligonucleotide
  source 1..24: mol_type = other DNA
gggtccattatttgagactttcca

SEQ ID NO: 35 - DNA, 25 bp, synthetic construct
  misc_feature 1..25: note = Synthetic Oligonucleotide
  source 1..25: mol_type = other DNA
agctttctgctggccccattacttg

SEQ ID NO: 36 - DNA, 19 bp, synthetic construct
  misc_feature 1..19: note = Synthetic Oligonucleotide
  source 1..19: mol_type = other DNA
gcatgtgcctgtcttcaca

SEQ ID NO: 37 - DNA, 25 bp, synthetic construct
  misc_feature 1..25: note = Synthetic Oligonucleotide
  source 1..25: mol_type = other DNA
gagtggtacaacctctacatttgag

SEQ ID NO: 38 - DNA, 23 bp, synthetic construct
  misc_feature 1..23: note = Synthetic Oligonucleotide
  source 1..23: mol_type = other DNA
ctgcagccttccctggttggttc

SEQ ID NO: 39 - DNA, 20 bp, synthetic construct
  misc_feature 1..20: note = Synthetic Oligonucleotide
  source 1..20: mol_type = other DNA
agtgggcagctccctttaga

SEQ ID NO: 40 - DNA, 19 bp, synthetic construct
  misc_feature 1..19: note = Synthetic Oligonucleotide
  source 1..19: mol_type = other DNA
cccactgcatgtgaagcat

SEQ ID NO: 41 - DNA, 22 bp, synthetic construct
  misc_feature 1..22: note = Synthetic Oligonucleotide
  source 1..22: mol_type = other DNA
ttgagtgtctgcacctgagcca

SEQ ID NO: 42 - DNA, 23 bp, synthetic construct
  misc_feature 1..23: note = Synthetic Oligonucleotide
  source 1..23: mol_type = other DNA
ggagacctcttattagcctgtga

SEQ ID NO: 43 - DNA, 20 bp, synthetic construct
  misc_feature 1..20: note = Synthetic Oligonucleotide
  source 1..20: mol_type = other DNA
cacctgccctaagtcatctc

SEQ ID NO: 44 - DNA, 22 bp, synthetic construct
  misc_feature 1..22: note = Synthetic Oligonucleotide
  source 1..22: mol_type = other DNA
tacagcaagagcagaggccaca

SEQ ID NO: 45 - DNA, 21 bp, synthetic construct
  misc_feature 1..21: note = Synthetic Oligonucleotide
  source 1..21: mol_type = other DNA
gcctgcagagaagcaatgttc

SEQ ID NO: 46 - DNA, 19 bp, synthetic construct
  misc_feature 1..19: note = Synthetic Oligonucleotide
  source 1..19: mol_type = other DNA
cagccgctacacaccacaa

SEQ ID NO: 47 - DNA, 24 bp, synthetic construct
  misc_feature 1..24: note = Synthetic Oligonucleotide
  source 1..24: mol_type = other DNA
ccagctgctacacaaatgcagggc

SEQ ID NO: 48 - DNA, 20 bp, synthetic construct
  misc_feature 1..20: note = Synthetic Oligonucleotide
  source 1..20: mol_type = other DNA
cgtcatctcctgccagttca

SEQ ID NO: 49 - DNA, 21 bp, synthetic construct
  misc_feature 1..21: note = Synthetic Oligonucleotide
  source 1..21: mol_type = other DNA
ctgctgctcagagcattgaaa

SEQ ID NO: 50 - DNA, 23 bp, synthetic construct
  misc_feature 1..23: note = Synthetic Oligonucleotide
  source 1..23: mol_type = other DNA
ccatggccagggaaggcttacta

SEQ ID NO: 51 - DNA, 22 bp, synthetic construct
  misc_feature 1..22: note = Synthetic Oligonucleotide
  source 1..22: mol_type = other DNA
cgcctccgactgaacatatgac

SEQ ID NO: 52 - DNA, 25 bp, synthetic construct
  misc_feature 1..25: note = Synthetic Oligonucleotide
  source 1..25: mol_type = other DNA
cctgatatgcatctttccctatgga

SEQ ID NO: 53 - DNA, 23 bp, synthetic construct
  misc_feature 1..23: note = Synthetic Oligonucleotide
  source 1..23: mol_type = other DNA
atggcacctcagaccagacccac

SEQ ID NO: 54 - DNA, 24 bp, synthetic construct
  misc_feature 1..24: note = Synthetic Oligonucleotide
  source 1..24: mol_type = other DNA
ggcagcaataatactgggacaaac

SEQ ID NO: 55 - DNA, 21 bp, synthetic construct
  misc_feature 1..21: note = Synthetic Oligonucleotide
  source 1..21: mol_type = other DNA
ggccaggaaacacatctgaag

SEQ ID NO: 56 - DNA, 27 bp, synthetic construct
  misc_feature 1..27: note = Synthetic Oligonucleotide
  source 1..27: mol_type = other DNA
agcagtgactctaaatgctcagtgtga

SEQ ID NO: 57 - DNA, 23 bp, synthetic construct
  misc_feature 1..23: note = Synthetic Oligonucleotide
  source 1..23: mol_type = other DNA
gcctcactccaacaacattatga